(19) United States
(12) Patent Application Publication
(10) Pub. No.: US 2025/0022557 A1
(43) Pub. Date: Jan. 16, 2025

(54) MEDICAL SYSTEM, CONTROL METHOD THEREOF, AND NON-TRANSITORY COMPUTER-READABLE STORAGE MEDIUM

(71) Applicant: CANON KABUSHIKI KAISHA, Tokyo (JP)

(72) Inventor: MUNETAKA SAKATA, Tokyo (JP)

(21) Appl. No.: 18/758,149

(22) Filed: Jun. 28, 2024

(30) Foreign Application Priority Data
Jul. 13, 2023 (JP) 2023-115465

(51) Int. Cl. G16H 10/60; A61B 5/00

(52) U.S. Cl. CPC G16H 10/60; A61B 5/0013; A61B 5/444

(57) ABSTRACT

A medical system that includes an electronic medical record terminal and an image capture apparatus that includes image capture unit, the system comprising: a specifying unit configured to specify a photographing person of the image capture apparatus; an obtaining unit configured to obtain patient information regarding a patient whose medical record information is to be displayed on the electronic medical record terminal that is being operated by the photographing person specified by the specifying unit; and a unit configured to associate a lesion image obtained by the image capture unit performing image capturing and the patient information obtained by the obtaining unit with each other.

### BACKGROUND OF THE INVENTION

### Field of the Invention

The present invention relates to a medical system, a control method thereof, and a non-transitory computer-readable storage medium.

### Description of the Related Art

In the field of medicine, images of lesions of patients are captured by digital cameras, and the images of the lesions are kept as treatment records and medical examination records. In order to keep an image of a lesion as a treatment record and a medical examination record, a photographing person needs to associate the image of the lesion with patient information such as a patient name and gender written in electronic medical records. Specifically, a photographing person logs into an electronic medical record terminal, displays medical record information of a patient whose image is to be captured, performs identity confirmation, and then captures an image of a lesion. The photographing person then performs an operation of associating patient information displayed on the electronic medical record terminal with the image of the lesion. Japanese Patent Laid-Open No. 2006-254957 discloses a method for authenticating an operator of an X-ray diagnosis apparatus, and permitting the operator to perform an operation, for example.

However, with a conventional technology disclosed in the above patent literature, it is possible to authenticate and specify an operator of an X-ray diagnosis apparatus that is an image capture apparatus, but it is not possible to obtain patient information and associate the patient information with an image of a lesion.

### SUMMARY OF THE INVENTION

In view of this, the present invention provides an apparatus that associates an image of a lesion shot by a medical worker with patient information of a patient.

According to one aspect of the present disclosure, there is provided a medical system that includes an electronic medical record terminal and an image capture apparatus that includes image capture unit, the system comprising: a specifying unit configured to specify a photographing person of the image capture apparatus; an obtaining unit configured to obtain patient information regarding a patient whose medical record information is to be displayed on the electronic medical record terminal that is being operated by the photographing person specified by the specifying unit; and a unit configured to associate a lesion image obtained by the image capture unit performing image capturing and the patient information obtained by the obtaining unit with each other.

According to another aspect of the present disclosure, there is provided a control method of a medical system that includes an electronic medical record terminal and an image capture apparatus that includes an image capture unit, the method comprising: specifying a photographing person of the image capture apparatus; obtaining patient information regarding a patient whose medical record information is to be displayed on the electronic medical record terminal that is being operated by the photographing person specified in the specifying; and associating a lesion image obtained by the image capture unit performing image capturing and the patient information obtained in the obtaining, to each other.

According to one aspect of the present disclosure, there is provided a non-transitory computer-readable storage medium storing thereon a computer program which, when loaded into and executed by a computer provided in a medical system that includes an electronic medical record terminal and an image capture apparatus that includes an image capture unit, causes the computer to perform processes comprising: specifying a photographing person of the image capture apparatus; obtaining patient information regarding a patient whose medical record information is to be displayed on the electronic medical record terminal that is being operated by the photographing person specified in the specifying; and associating a lesion image obtained by the image capture unit performing image capturing and the patient information obtained in the obtaining, to each other.

Further features of the present invention will become apparent from the following description of exemplary embodiments (with reference to the attached drawings).

### BRIEF DESCRIPTION OF THE DRAWINGS

FIG. 1 is a diagram of a system configuration of an image capture system.

FIG. 2 is a block diagram of an image capture apparatus.

FIG. 3 is a block diagram of an information processing apparatus.

FIG. 4 is a diagram showing the image capture apparatus as viewed from the rear side.

FIG. 5A is a display screen of an image capture apparatus according to a first embodiment.

FIG. 5B is a display screen of the image capture apparatus according to the first embodiment.

FIG. 5C is a display screen of the image capture apparatus according to the first embodiment.

FIG. 6A is a patient information table of an electronic medical record management apparatus according to the first embodiment.

FIG. 6B is a medical worker information table of the electronic medical record management apparatus according to the first embodiment.

FIG. 6C is a clinical examination patient information table of the electronic medical record management apparatus according to the first embodiment.

FIG. 7A is a diagram of a processing sequence of an image capture system according to the first embodiment.

FIG. 7B is a diagram of a processing sequence of an image capture system according to the first embodiment.

FIG. 8A is a flowchart of the image capture apparatus according to the first embodiment.

FIG. 8B is a flowchart of an image capture apparatus management apparatus according to the first embodiment.

FIG. 8C is a flowchart of an electronic medical record terminal apparatus according to the first embodiment.

FIG. 8D is a flowchart of the electronic medical record management apparatus according to the first embodiment.

FIG. 9A shows an operation example of the first embodiment.

FIG. 9B shows an operation example of the first embodiment.

FIG. 9C shows an operation example of the first embodiment.

FIG. 9D shows an operation example of the first embodiment.

FIG. 10A is a block diagram of an image capture apparatus according to a second embodiment.

FIG. 10B is a display screen of the image capture apparatus according to the second embodiment.

FIG. 11 is a diagram of a processing sequence when an image of a patient ID barcode is captured, in an image capture system according to the second embodiment.

FIG. 12A is a flowchart of the image capture apparatus according to the second embodiment.

FIG. 12B is a flowchart of the image capture apparatus according to the second embodiment.

FIG. 13A is a diagram of a processing sequence of an image capture system according to a third embodiment.

FIG. 13B is a diagram of a processing sequence of an image capture system according to a third embodiment.

FIG. 14A is a flowchart of an image capture apparatus according to the third embodiment.

FIG. 14B is a flowchart of an image capture apparatus according to the third embodiment.

### DESCRIPTION OF THE EMBODIMENTS

Hereinafter, embodiments will be described in detail with reference to the attached drawings. Note, the following embodiments are not intended to limit the scope of the claimed invention. Multiple features are described in the embodiments, but limitation is not made to an invention that requires all such features, and multiple such features may be combined as appropriate. Furthermore, in the attached drawings, the same reference numerals are given to the same or similar configurations, and redundant description thereof is omitted.

### First Embodiment

First, an overview of an image capture system according to an embodiment will be described. The image capture system according to the embodiment is a medical image capture system. The image capture system includes an image capture apparatus and an image capture apparatus control apparatus as apparatuses that constitute the system. When a medical worker is authenticated by the image capture apparatus, the image capture apparatus, which also functions as a photographing person specifying unit, specifies the authenticated medical worker as a photographing person, obtains patient information of a patient whose image is to be shot by the medical worker, from an electronic medical record management apparatus, and sets the patient information in the image capture apparatus. After that, when the medical worker shoots an image of a lesion of the patient using the image capture apparatus, the image of the lesion is stored in the image capture apparatus in association with the patient information. When the medical worker completes lesion image capturing, the image capture apparatus transmits the image of the lesion associated with the patient information to the image capture apparatus control apparatus. Furthermore, the image capture apparatus control apparatus transmits the received image of the lesion to an image management apparatus.

In addition, the image capture system according to the embodiment includes an electronic medical record terminal apparatus, the electronic medical record management apparatus, and the image management apparatus, as apparatuses that constitute the system. When a medical worker logs into the electronic medical record terminal apparatus using login information and the like, medical record information (patient information and patient clinical examination information) of a patient stored in the electronic medical record management apparatus can be displayed on the electronic medical record terminal apparatus. In addition, the electronic medical record management apparatus can read a lesion image stored in the image management apparatus, and displays a lesion image on the electronic medical record terminal apparatus when the electronic medical record terminal apparatus makes a request to display the lesion image. That is to say, in the image capture system according to the embodiment, an image of a lesion captured by the image capture apparatus is stored in the image management apparatus. The medical worker then displays the image of the lesion stored in the image management apparatus, on a display of the electronic medical record terminal apparatus, and uses the image for clinical examination of the patient.

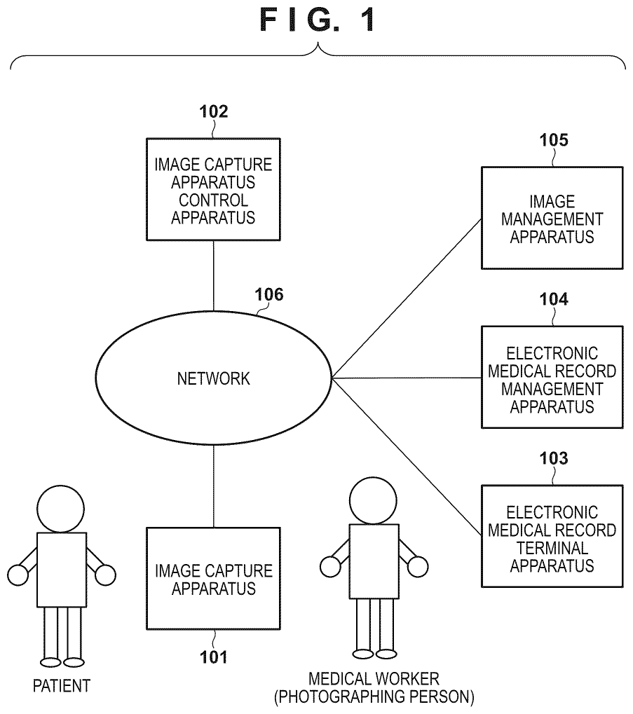

FIG. 1 is a diagram of a system configuration of the image capture system according to the embodiment. The image capture system is an example of a medical system. An image capture apparatus control apparatus 102, an electronic medical record management apparatus 104, and an image management apparatus 105 are examples of management apparatuses. An image capture apparatus 101 and an electronic medical record terminal apparatus 103 are examples of terminal systems.

The image capture apparatus 101 is an apparatus that captures an image of a subject and generates image data. The image capture apparatus 101 is a small-sized portable image capture apparatus, for example, as with a commercially available digital camera, and includes a display device that enables a captured image to be confirmed, and interfaces such as buttons and a touch panel for performing various operations. In the present embodiment, a description will be given assuming that the image capture apparatus 101 is a digital camera, and a medical worker such as a doctor or a clinical nurse serves as a photographing person, and shoots an image of a lesion of a patient using the image capture apparatus 101 to generate a lesion image. The image capture apparatus 101 transmits, as a set, the lesion image and patient information, which is information regarding the patient.

The image capture apparatus control apparatus 102 is an information processing apparatus such as a computer, and is an apparatus that intermediates communication between the image capture apparatus 101, the electronic medical record management apparatus 104, and the image management apparatus 105. The image capture apparatus control apparatus 102, which functions as a portion of a patient information transmitting unit, obtains medical worker information and patient information from the electronic medical record management apparatus 104 and transmits the information to the image capture apparatus 101, and transmits a lesion image to the image management apparatus 105.

The electronic medical record terminal apparatus 103 is, for example, an information processing apparatus, and operates as an electronic medical record display device by a medical worker performing a login operation. The electronic medical record terminal apparatus 103 can download patient information from the electronic medical record management apparatus 104 and display the patient information as an electronic medical record, and can upload information updated by the electronic medical record terminal apparatus 103, to the electronic medical record management apparatus 104. In the present embodiment, a medical worker such as a doctor or a clinical nurse performs an operation on the electronic medical record terminal apparatus 103, to display a captured image of a lesion of a patient, and write a clinical examination result such as a disease condition in an electronic medical record.

The electronic medical record management apparatus 104 is, for example, an information processing apparatus, and is an apparatus that stores patient information (patient ID, name, age, and gender), patient clinical examination information (patient ID, diagnosis and treatment department, disease condition, past medical history, etc.), and medical worker information (medical worker ID, name, and authentication information such as password). The patient ID is an example of patient identification information. The medical worker ID is also referred to as “medical worker identification information”. In addition, the electronic medical record management apparatus 104 authenticates a request to authenticate a medical worker from the image capture apparatus control apparatus 102 or the electronic medical record terminal apparatus 103, and provides information in response to a request to obtain patient information and medical worker information. Furthermore, the electronic medical record management apparatus 104 receives information updated by the electronic medical record terminal apparatus 103, and updates and stores patient information and medical worker information.

The image management apparatus 105 is, for example, an information processing apparatus, and is an apparatus that stores lesion images, photographing person information associated with the lesion images, and patient information associated with the lesion images.

A network 106 is an inter-device network for performing wireless data transmission and by-wire data transmission, various types of control, and the like. Various communication protocols such as the hypertext transfer protocol (HTTP), the file transfer protocol (FTP), the picture transfer protocol (PTP), a universal serial bus (USB), a local area network (LAN), health level seven (HL7), and digital imaging and communications in medicine (DICOM) are conceivable, but any of those may be used as long as image data, text, control data, and the like can be transmitted/received.

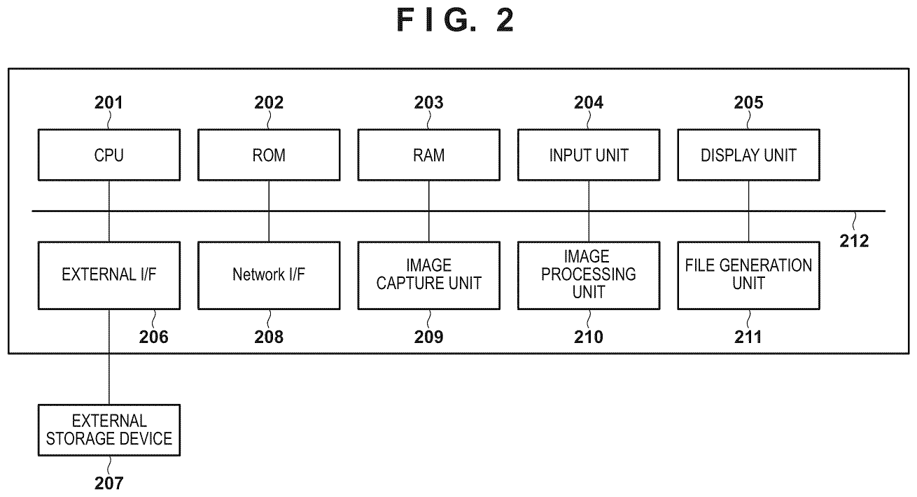

FIG. 2 is a block diagram of the image capture apparatus 101 according to the embodiment. Image capture processing to be described later is realized by the image capture apparatus 101 executing a predetermined control program, which functions as an image capture apparatus.

A CPU 201 performs overall control of the image capture apparatus 101.

A ROM 202 is a ROM that stores operation processing procedures of the CPU 201 (programs of processing performed when the power source of the image capture apparatus 101 is turned on, and programs of basic input/output processing and the like).

A RAM 203 is a random access memory (RAM), and functions as a main memory of the CPU 201, but another memory device may be used. The RAM 203 may be constituted by a non-volatile memory device such as a read only memory (ROM) or an electrically erasable programmable read-only memory (EEPROM). Various programs that include a control program for realizing later-described processing are loaded from the ROM 202 or the like to the RAM 203, and are executed by the CPU 201. In addition, the RAM 203 provides a work area that is used when the CPU 201 executes various types of processing.

An input unit 204 is an input unit such as buttons for performing various operations. The input unit 204 includes, for example, a release button positioned on the upper side of a digital camera, a cross key positioned on a rear surface portion, a setting key, and the like. In addition, a touch panel provided on a display unit 205 and the like are also included in an input device. A user (photographing person) can input various instructions to a digital camera (the image capture apparatus 101) by performing an operation on the input device through the input unit 204.

The display unit 205 is a display device, and performs various types of display under control of the CPU 201. The display unit 205 displays an image captured by an image capture unit 209 in accordance with an operation being performed on the release button to input an image capture instruction, various setting screens, and the like. In addition, the display unit 205 may display an image that is based on image data stored in the RAM 203 or an external storage device 207, and a live view image captured by the image capture unit 209.

An external I/F 206 is an I/F for reading/writing data from/to the external storage device 207 such as an SD card or a USB memory.

The external storage device 207 is an external storage medium such as an SD card or a USB memory.

A network I/F 208 is a network interface, and is connected to the network 106. In the present embodiment, the image capture apparatus 101 transmits/receives data to/from the image capture apparatus control apparatus 102 via the network 106.

The image capture unit 209 reads an optical image using a solid-state image sensor, performs analog-digital conversion on the optical image, and generate electric image data.

An image processing unit 210 executes various types of image processing. The CPU 201 stores, in the RAM 203, image data generated by the image capture unit 209 and attribute information at this time. The image processing unit 210 performs a series of image processing so as to obtain image data suitable for an image capturing purpose, such as data that reflects human visual feature, three-dimensional data, or a combination thereof, as necessary.

A file generation unit 211 executes file generation processing. The CPU 201 converts image data generated by the image processing unit 210 into a general-purpose still image format, and generates an image file. In addition, EXIF information can be added to and changes can be made to image data generated by the image processing unit 210 and an image file generated by the file generation unit 211.

A CPU bus 212 includes an address bus, a data bus, and a control bus. The CPU bus 212 connects the CPU 201, the ROM 202, the RAM 203, the input unit 204, the display unit 205, the external I/F 206, the network I/F 208, the image capture unit 209, the image processing unit 210, and the file generation unit 211 to each other.

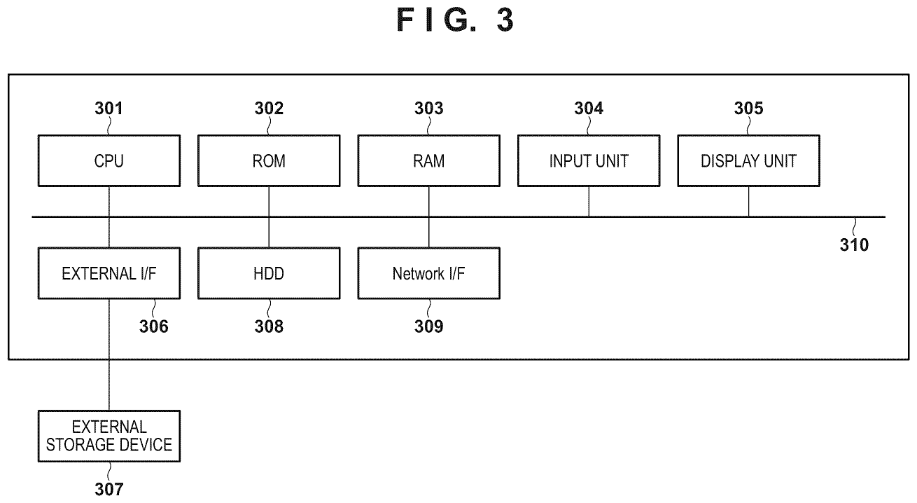

FIG. 3 is a block diagram of an information processing apparatus according to the embodiment. Later-described processing is performed by a predetermined control program being executed in the information processing apparatus.

In the embodiment, the image capture apparatus control apparatus 102, the electronic medical record terminal apparatus 103, the electronic medical record management apparatus 104, and the image management apparatus 105 are information processing apparatuses.

A CPU 301 performs overall control of the information processing apparatus.

A ROM 302 is a ROM that stores operation processing procedures (programs of processing of the information processing apparatus, basic input/output processing, and the like) of the CPU 301.

A RAM 303 is a random access memory (RAM), and functions as a main memory of the CPU 301, but another memory device may be used. The RAM 303 may be constituted by a non-volatile memory device such as a read only memory (ROM) or an electrically erasable programmable read-only memory (EEPROM). Various programs that include a control program for realizing later-described processing are loaded from the ROM 302 or the like to the RAM 303, and are executed by the CPU 301. In addition, the RAM 303 provides a work area that is used when the CPU 301 executes various types of processing.

An input unit 304 is a keyboard that includes various keys for inputting characters, or a pointing device such as a mouse. The input unit 304 is used for an operation of inputting setting information to a program, or an operation of selecting an icon, a menu, or the like that is displayed on a display unit 305, for example.

The display unit 305 is a display, and displays images for an interface to the operator, such as a window, icons, a message, and a menu.

An external I/F 306 is an I/F for reading/writing data from/to an external storage device 307 such as an external HDD or a USB memory.

The external storage device 307 is an external storage medium such as an external HDD or a USB memory.

An HDD 308 is a non-volatile storage device, and is a hard disk drive (HDD) that stores data.

A network I/F 309 is a network interface, and is connected to the network 106. In the present embodiment, the image capture apparatus 101, the image capture apparatus control apparatus 102, the electronic medical record terminal apparatus 103, the electronic medical record management apparatus 104, and the image management apparatus 105 are communicably connected to each other via the network 106, and transmit/receive data to each other.

A CPU bus 310 includes an address bus, a data bus, and a control bus. The CPU bus 310 connects the CPU 301, the ROM 302, the RAM 303, the input unit 304, the display unit 305, the external I/F 306, the HDD 308, and the network I/F 309 to each other.

Here, a supplementary description will be given on data stored in the electronic medical record management apparatus 104 according to the present embodiment. The electronic medical record management apparatus 104 stores, in the HDD 308, patient information (patient ID, name, age, and gender) and patient clinical examination information (patient ID, diagnosis and treatment department, disease condition, past medical history, etc.), and medical worker information (medical worker ID, name, and authentication information such as password).

The patient information is information for specifying a patient, and a unique patient ID is defined for each patient. In the present embodiment, the patient information is a patient information table (to be described later).

The patient clinical examination information is information obtained by recording disease conditions of patients in an accumulated manner. The patient clinical examination information is stored in a patient clinical examination information table (not illustrated) in association with patient IDs. For this reason, if a patient ID is known, the diagnosis and treatment department, the disease condition, the past medical history, and the like of the patient can be known. That is to say, electronic medical record information can be generated by combining patient information obtained from the patient information table and patient clinical examination information obtained from the patient clinical examination information table, using a patient ID as a key.

The medical worker information is information for specifying a medical worker, and a unique medical worker ID is defined for each medical worker. In the present embodiment, the medical worker information is a medical worker information table (to be described later).

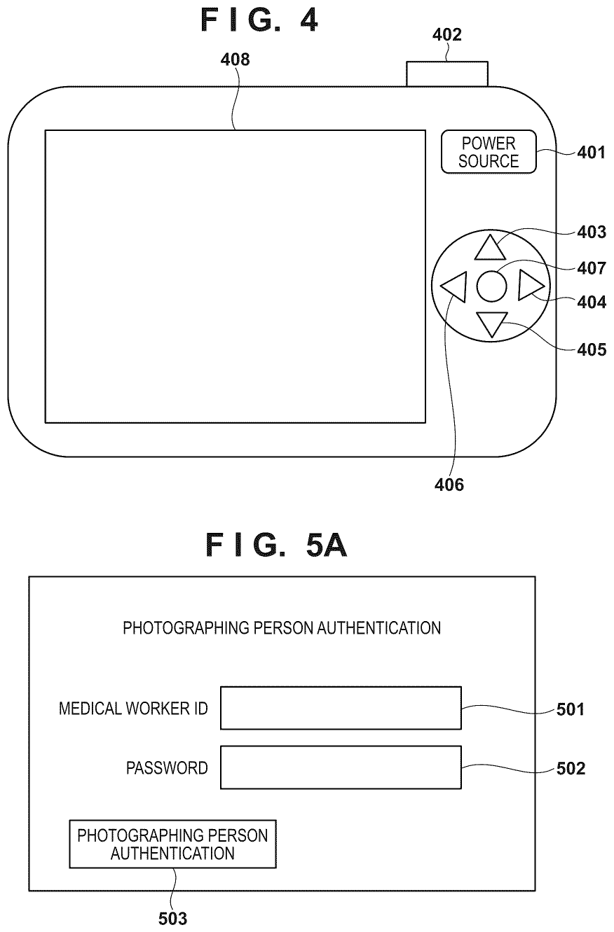

FIG. 4 is a diagram showing the image capture apparatus 101 as viewed from the rear side. The input unit 204 and the display unit 205 of the image capture apparatus 101 will be described with reference to FIG. 4.

The input unit 204 is constituted by a power source button 401, a release button 402, an up button 403, a right button 404, a down button 405, a left button 406, and a submit button 407.

The power source button 401 is a power source button for switching on/off the power source. When a photographing person presses the power source button 401 in a state where the power source of the image capture apparatus 101 is off, the CPU 201 determines that an instruction to turn on the power source has been given from the photographing person, and turns on the power source. When the photographing person presses the power source button 401 in a state where the power source is on, the CPU 201 determines that an instruction to turn on the power source has been given from the photographing person, and turns off the power source.

The release button 402 is a button that a photographing person presses when capturing an image of a subject. When the photographing person presses the release button 402, the CPU 201 determines that an image capture instruction to capture a still image or a moving image has been given, and generates an image of a subject using the image capture unit 209, the image processing unit 210, and the file generation unit 211. In the present embodiment, the subject is a lesion of a patient, and thus, when a photographing person presses the release button 402, an image of a lesion of a patient is generated.

The up button 403, the right button 404, the down button 405, the left button 406, and the submit button 407 are buttons that are used when a photographing person selects an item displayed on a display 408 and performs determination.

The display 408 functions as the display unit 205. The display 408 is a display device for displaying an input screen for prompting the photographing person to perform various types of input, and displaying a captured image.

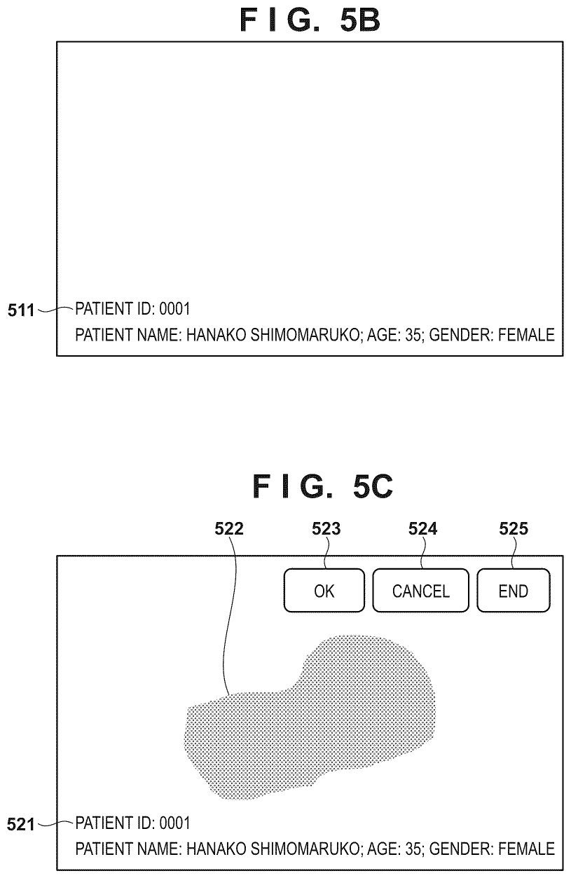

FIGS. 5A, 5B, and 5C show screens that are displayed on the display 408 of the image capture apparatus 101.

FIG. 5A shows a photographing person authentication screen. A medical worker ID input area 501 is an area for inputting an ID of a medical worker that shoots an image using the image capture apparatus 101. A password input area 502 is an area for inputting a password of the medical worker that shoots an image using the image capture apparatus 101. When an ID and a password are input, a software keyboard screen (not illustrated) is displayed so as to allow alphabets, numerals, and the like to be input. A photographing person authentication button 503 is a button that is pressed when photographing person authentication is performed using a medical worker ID input to the medical worker ID input area 501 and a password input to the password input area 502.

FIG. 5B shows a patient information display screen. In the patient information display screen, a patient information display area 511 is an area for displaying patient information. The patient ID, the name, the age, and the gender of a patient are displayed in the patient information display area 511.

FIG. 5C shows a lesion image display screen. In the lesion image display screen, a patient information display area 521, an OK button 523, a cancel button 524, and an end button 525 are displayed, and are superimposed on a captured image of a lesion. The patient information display area 521 is an area for displaying patient information similarly to the patient information display area 511. A lesion 522 is an area for a captured image of a lesion of a patient. The OK button 523 is a button that is pressed when the next lesion image is captured without deleting the lesion image displayed in the lesion image display screen. The cancel button 524 is a button that is pressed when the lesion image displayed in the lesion image display screen is deleted and the next lesion image is then captured. The end button 525 is a button that is pressed when lesion image capturing is ended.

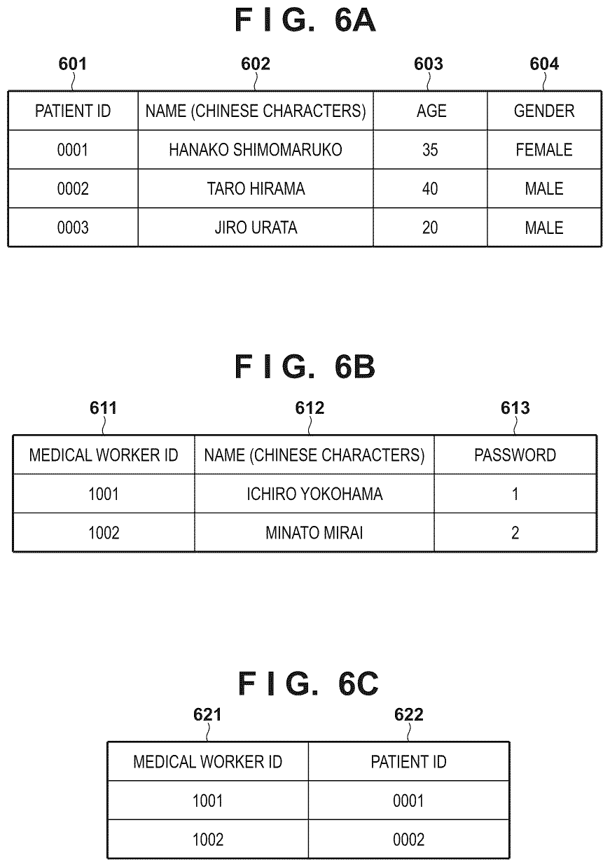

FIGS. 6A, 6B, and 6C show information tables that are held in the HDD 308 by the electronic medical record management apparatus 104.

FIG. 6A shows a patient information table. Information regarding patients is managed in the patient information table. The patient information table is composed of patient ID 601, name (Chinese characters) 602, age 603, and gender 604. The patient ID 601 is an ID composed of a four-digit number and enabling a patient to be identified, for example. The name (Chinese characters) 602 is a patient name written in Chinese characters. The age 603 is the age of a patient. The gender 506 is the gender of a patient. The patient information table stores records of patients. In the example in FIG. 6A, patient records of three IDs are registered. In FIG. 6A, for example, “Hanako Shimomaruko” is registered as a patient name corresponding to a patient ID “0001”, “35” is registered as an age, and “female” is registered as a gender.

FIG. 6B shows a medical worker information table. Information regarding medical workers is managed using the medical worker information table. The medical worker information table is composed of medical worker ID 611, name (Chinese character) 612, and password 613. The medical worker ID 611 is an ID composed of a four-digit number and enabling a medical worker to be identified, for example. The name (Chinese character) 612 is a medical worker name written in Chinese characters. The password 613 is a password corresponding to the medical worker ID. The medical worker information table stores records for each medical worker. In the example in FIG. 6B, medical worker records of two IDs are registered. In FIG. 6B, for example, the name of a medical worker whose medical worker ID is 1001 is Ichiro Yokohama, and 1 is registered as his password.

FIG. 6C shows a clinical examination patient information table. The clinical examination patient information table is a table for managing patients on which a medical worker is performing clinical examination. The clinical examination patient information table is composed of medical worker ID 621 and patient ID 622. The medical worker ID 621 is an ID composed of a four-digit number and enabling a medical worker to be identified, an ID registered as the medical worker ID 611 in the medical worker information table is registered as the medical worker ID 621. The patient ID 622 is an ID composed of a four-digit number and enabling a patient to be identified, and an ID registered as the patient ID 601 in the patient information table is registered as the patient ID 622. In the example in FIG. 6C, two records are registered. In FIG. 6C, for example, it can be seen that a medical worker whose medical worker ID is 1001 is performing clinical examination on a patient whose patient ID is 0001.

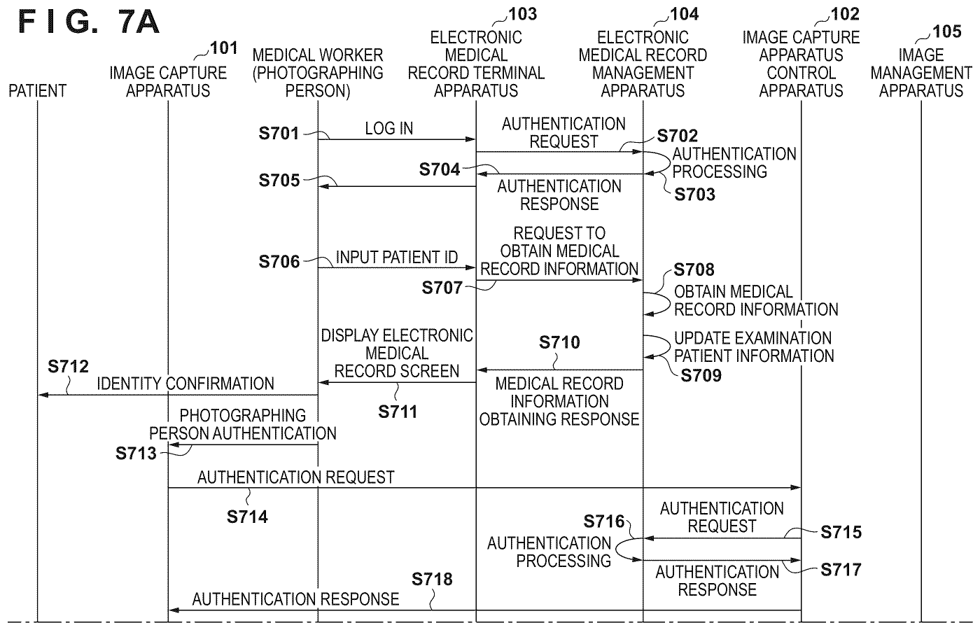

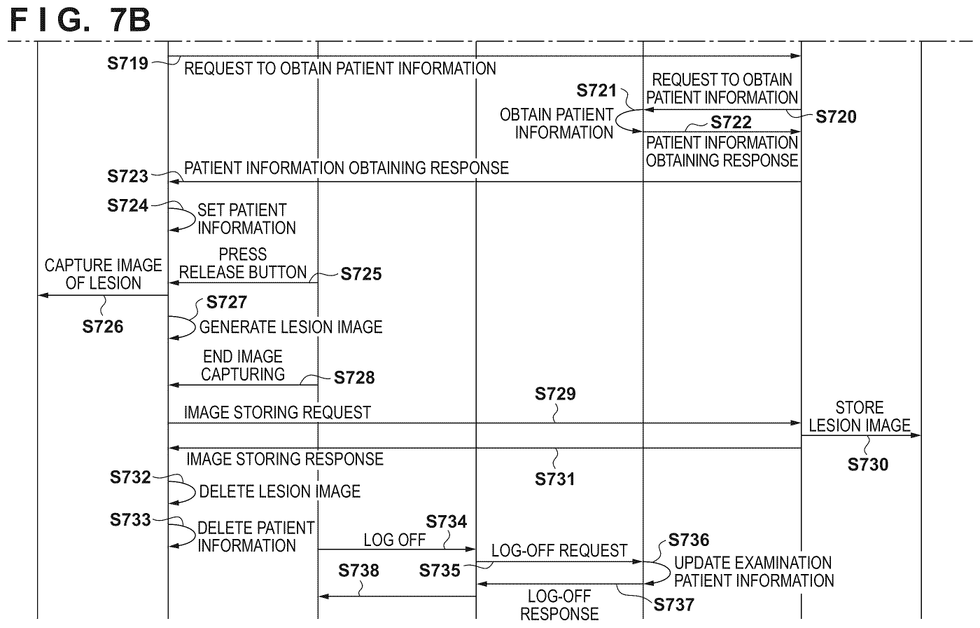

FIGS. 7A and 7B is a diagram of a processing sequence of the image capture system according to the present embodiment. Based on the illustration in FIGS. 7A and 7B, a description will be given assuming that the medical worker is a medical practitioner. FIGS. 7A and 7B illustrates a flow in which, when a medical practitioner logs into an electronic medical record terminal apparatus, displays the electronic medical record of a patient, and shoots an image of a lesion of the patient using an image capture apparatus, the patient on which the medical practitioner that is a photographing person is performing clinical examination is specified, and the image capture apparatus stores, in an image management apparatus, the lesion image and patient information in association with each other.

Steps S701 to S711 represent a processing sequence from when a medical practitioner that is a medical worker logs into the electronic medical record terminal apparatus 103 until when the electronic medical record of a patient is displayed.

In step S701, the medical practitioner inputs a medical worker ID and a password to the electronic medical record terminal apparatus 103 and performs login. At this time, the electronic medical record terminal apparatus 103 may accept the medical worker ID and the password from the medical practitioner in a state where the photographing person authentication screen shown in FIG. 5A is displayed, for example.

In step S702, the electronic medical record terminal apparatus 103 transmits a request for authentication of the medical worker ID and the password input by the medical practitioner (login authentication), to the electronic medical record management apparatus 104.

In step S703, the electronic medical record management apparatus 104 performs authentication processing in accordance with the received authentication request. In authentication processing, the electronic medical record management apparatus 104 determines whether or not the combination of the medical worker ID and the password, which are included in the medical worker information table shown in FIG. 6B, is correct.

In step S704, if the combination of the medical worker ID and the password is correct, the electronic medical record management apparatus 104 transmits an authentication response indicating that authentication was successfully performed, to the electronic medical record terminal apparatus 103. If the combination of the medical worker ID and the password is not correct, the electronic medical record management apparatus 104 transmits an authentication response indicating that authentication failed, to the electronic medical record terminal apparatus 103.

In step S705, if the authentication response indicates that authentication was successfully performed, the electronic medical record terminal apparatus 103 displays an electronic medical record screen. If the authentication response indicates that authentication failed, the electronic medical record terminal apparatus 103 displays a screen for inputting a medical worker ID and a password again, and waits for the medical practitioner to log in (the procedure returns step S701).

In step S706, the medical practitioner inputs, to the electronic medical record terminal apparatus 103, information that makes it possible to identify a patient on which clinical examination is to be performed. Here, the information that makes it possible to identify a patient is a patient ID included in the patient information table shown in FIG. 6A.

In step S707, the electronic medical record terminal apparatus 103 transmits, to the electronic medical record management apparatus 104, a request to obtain medical record information corresponding to the patient ID input by the medical practitioner.

In step S708, the electronic medical record management apparatus 104 performs processing for obtaining medical record information, in accordance with the received request to obtain medical record information. In processing for obtaining medical record information, the electronic medical record management apparatus 104 performs processing for generating a disease condition that includes the patient ID, the name, the age, the gender, and the like included in the patient information table shown in FIG. 6A, as electronic medical record information.

In step S709, the electronic medical record management apparatus 104 registers, to the clinical examination patient information table in FIG. 6C, the medical worker ID of the medical practitioner and the patient ID of the patient as a pair. Note that, if there are already a record of the medical worker ID, the electronic medical record management apparatus 104 updates the patient ID only. That is to say, the clinical examination patient information table is data that makes it possible to specify a patient ID of a patient on which a medical practitioner is performing clinical examination, if a medical worker ID of the medical practitioner is known.

In step S710, the electronic medical record management apparatus 104 transmits, to the electronic medical record terminal apparatus 103, the electronic medical record information of the patient generated in the processing of step S708 as a medical record information obtaining response.

In step S711, the electronic medical record terminal apparatus 103 forms the received medical record information obtaining response, into a display format of electronic medical record, and displays the electronic medical record screen.

Steps S712 to S724 represent a processing sequence from when the medical practitioner confirms the identity of the patient until when the patient ID 601, the name (Chinese character) 602, the age 603, and the gender 604 included in the patient information table are set in the image capture apparatus 101.

In step S712, the medical practitioner performs identity confirmation, that is, confirms that the electronic medical record information displayed on the electronic medical record terminal apparatus 103 in step S711 is the electronic medical record information of the patient himself or herself. If the electronic medical record information is not the electronic medical record information of the patient, the medical practitioner inputs, to the electronic medical record terminal apparatus 103, information regarding the patient who is to be subjected to clinical examination, again (the procedure returns to step S706), and the electronic medical record terminal apparatus 103 displays electronic medical record information of the patient newly obtained from the electronic medical record management apparatus 104. When the electronic medical record information is the electronic medical record information of the patient, the medical practitioner advances the procedure to step S713.

In step S713, the medical practitioner inputs the medical worker ID and the password respectively to the medical worker ID input area 501 and the password input area 502 of the photographing person authentication screen in FIG. 5A that is displayed on the display unit of the image capture apparatus 101, and presses the photographing person authentication button 503.

In step S714, the image capture apparatus 101 transmits, to the image capture apparatus control apparatus 102, a request to authenticate the medical worker ID and the password input by the medical practitioner.

In step S715, the image capture apparatus control apparatus 102 transmits the received authentication request to the electronic medical record management apparatus 104.

In step S716, the electronic medical record management apparatus 104 performs authentication processing in accordance with the received authentication request. In authentication processing, the electronic medical record management apparatus 104 determines whether or not the combination of the medical worker ID and the password, which are included in the medical worker information table in FIG. 6B, is correct.

In step S717, if the combination of the medical worker ID and the password is correct, the electronic medical record management apparatus 104 transmits, to the image capture apparatus control apparatus 102, an authentication response indicating that authentication was successfully performed. If the combination of the medical worker ID and the password is not correct, the electronic medical record management apparatus 104 transmits, to the image capture apparatus control apparatus 102, an authentication response indicating that authentication failed.

In step S718, the image capture apparatus control apparatus 102 transmits the received authentication response to the image capture apparatus 101. The image capture apparatus 101 that has received the authentication response then advances the procedure to step S719 if the authentication response indicates that authentication was successfully performed. If the authentication response indicates that authentication failed, the image capture apparatus 101 displays a screen for inputting a medical worker ID and a password again, and waits for the medical practitioner to input a medical worker ID and a password (the procedure returns to step S713).

In step S719, the image capture apparatus 101 transmits, to the image capture apparatus control apparatus 102, a request to obtain patient information corresponding to the medical worker ID input by the medical practitioner in step S713.

In step S720, the image capture apparatus control apparatus 102 transmits the received request to obtain patient information, to the electronic medical record management apparatus 104.

In step S721, the electronic medical record management apparatus 104 performs processing for obtaining patient information, in accordance with the received request to obtain patient information. In processing for obtaining patient information, the electronic medical record management apparatus 104 obtains the medical worker ID included in the request to obtain patient information, and obtains a patient ID corresponding to the medical worker ID, from the clinical examination patient information table in FIG. 6C. The electronic medical record management apparatus 104 then obtains patient information associated with the obtained patient ID from the patient information table in FIG. 6A.

In step S722, the electronic medical record management apparatus 104 transmits, to the image capture apparatus control apparatus 102, a patient information obtaining response that includes the patient information obtained in step S721.

In step S723, the image capture apparatus control apparatus 102 transmits the received patient information obtaining response that includes the patient information, to the image capture apparatus 101.

In step S724, the image capture apparatus 101 obtains the patient information from the patient information obtaining response, and sets the patient information in the image capture apparatus 101.

Steps S725 to S733 represent a processing sequence from when the medical practitioner captures an image of a lesion of the patient until when the image capture apparatus 101 associates the captured lesion image and the patient information with each other, and transmits the captured lesion image and the patient information to the image management apparatus 105.

In step S725, the medical practitioner turns the image capture apparatus 101 toward the lesion of the patient, and presses the release button 402 of the image capture apparatus 101.

In step S726, the image capture apparatus 101 captures an image of the lesion of the patient, and generates lesion image data.

In step S727, the image capture apparatus 101 performs processing for associating the patient information obtained by the image capture apparatus 101 in step S724 and the lesion image data generated in step S726, with each other. In the present embodiment, the image capture apparatus 101 converts the lesion image data into a JPEG image, and sets the patient information (patient ID, patient name, etc.) in EXIF information of the JPEG image.

In step S728, the medical practitioner presses the end button 525 displayed on the display unit 205 of the image capture apparatus 101 in order to end lesion image capturing.

In step S729, the image capture apparatus 101 transmits a request to store the lesion image associated with the patient information in step S727, to the image capture apparatus control apparatus 102.

In step S730, the image capture apparatus control apparatus 102 performs processing in accordance with the received image storing request. The image capture apparatus control apparatus 102 stores the lesion image included in the image storing request, in the image management apparatus 105.

In step S731, when the lesion image is stored in the image management apparatus 105, the image capture apparatus control apparatus 102 transmits, to the image capture apparatus 101, an image storing response indicating that the image was successfully stored. Note that, if the lesion image could not be stored in the image management apparatus 105, the image capture apparatus control apparatus 102 transmits, to the image capture apparatus 101, an image storing response indicating that storing the image failed. If the image storing response indicates that the image was successfully stored, the image capture apparatus 101 performs processing of step S732. In addition, if the image storing response indicates that storing the image failed, the image capture apparatus 101 makes an image storing request again (the procedure returns to step S729).

In step S732, the image capture apparatus 101 deletes the patient information set in step S724.

In step S733, the image capture apparatus 101 deletes the lesion image transmitted in step S729.

Steps S734 to S738 represent a processing sequence in a case where the medical practitioner, who is a medical worker, logs off from the electronic medical record terminal apparatus 103, and the medical practitioner ends clinical examination of the patient.

In step S734, the medical practitioner logs off from the electronic medical record terminal apparatus 103.

In step S735, the electronic medical record terminal apparatus 103 transmits a log-off request for the medical worker ID of the medical worker that has logged in, to the electronic medical record management apparatus 104.

In step S736, the electronic medical record management apparatus 104 performs processing for updating the clinical examination patient information in accordance with the received log-off request. In processing for updating the clinical examination patient information, the electronic medical record management apparatus 104 deletes the record of the medical worker ID included in the log-off request, from the clinical examination patient information table in FIG. 6C. That is to say, the medical practitioner enters a state of not performing clinical examination of any patient.

In step S737, the electronic medical record management apparatus 104 transmits a log-off response to the electronic medical record terminal apparatus 103.

In step S738, upon receiving the log-off response, the electronic medical record terminal apparatus 103 waits for the medical practitioner to input a medical worker ID and a password (the procedure returns to step S701).

The description of the processing sequence in FIGS. 7A and 7B will be supplemented. For simplification, a description has been given assuming that, in the processing sequence in FIGS. 7A and 7B, an image of a lesion is captured one time, but an image of a lesion may be captured a plurality of times. In that case, after processing of steps S726 and S727 is repeated the number of times that an image of a lesion is captured, processing of step S728 is repeated.

The description of the processing sequence in FIGS. 7A and 7B will be supplemented. For simplification, a description has been given assuming that there is one patient in the processing sequence in FIGS. 7A and 7B, but, during a period from login to the electronic medical record terminal apparatus 103 until log-off, the medical practitioner may perform clinical examination on a plurality of patients. In that case, after processing of steps S706 to S733 is performed for each patient, processing of step S734 is performed.

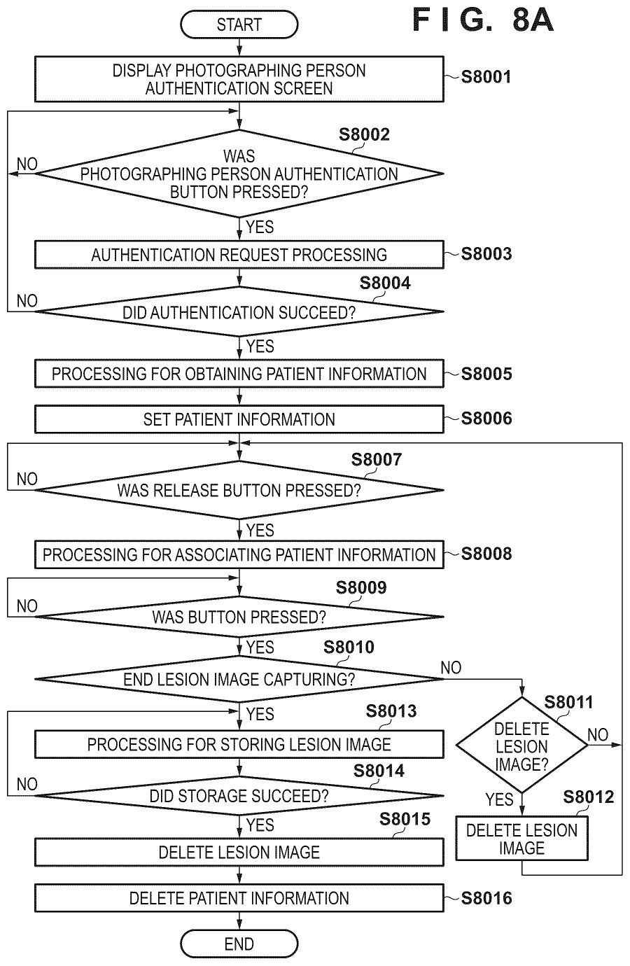

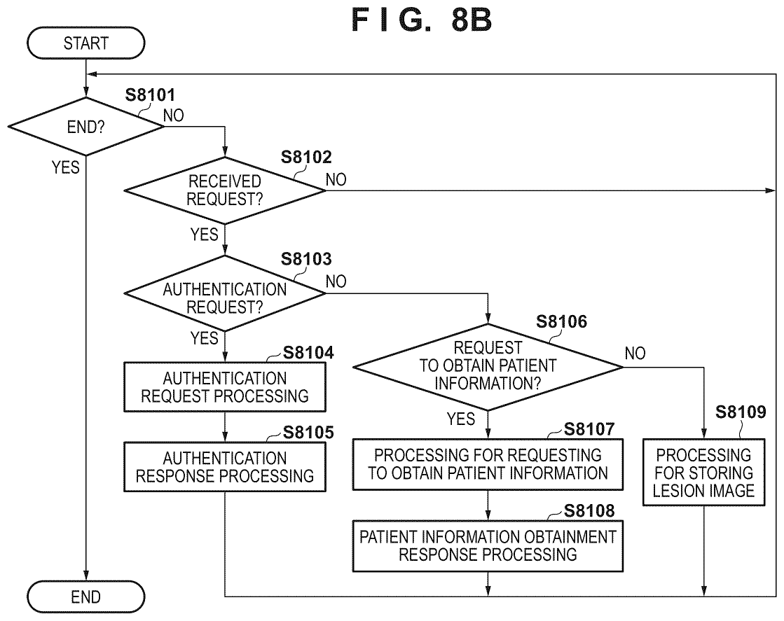

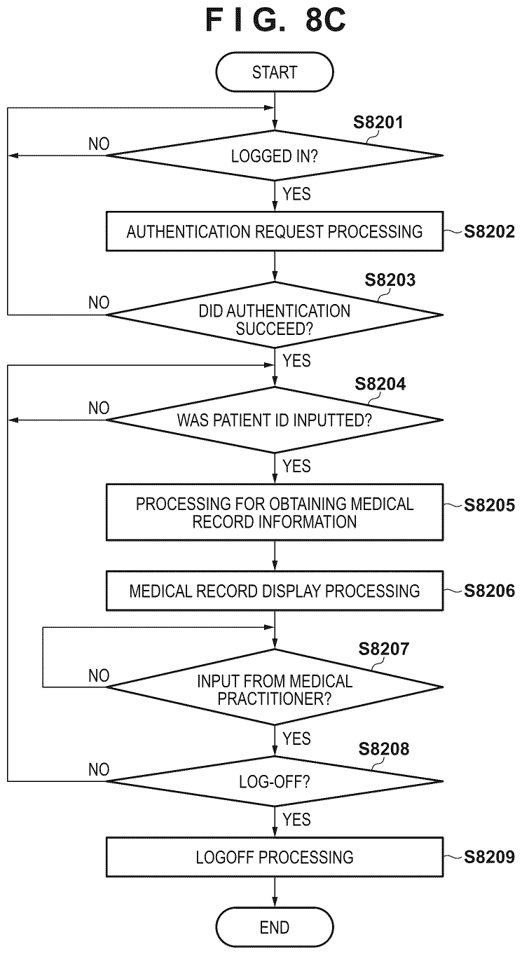

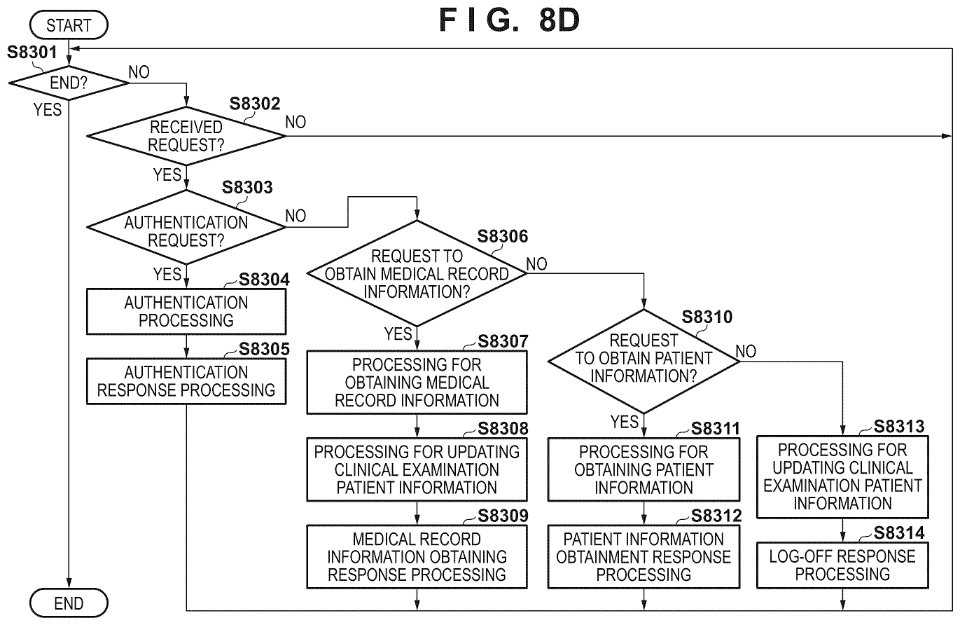

FIGS. 8A, 8B, 8C, and 8D are flowcharts of the image capture apparatus 101, the image capture apparatus control apparatus 102, the electronic medical record terminal apparatus 103, and the electronic medical record management apparatus 104, respectively, for realizing the processing sequence of the image capture system described with reference to FIGS. 7A and 7B.

Description will be given with reference to FIG. 8A. Processing of the flowchart in FIG. 8A is stored in the ROM 202 of the image capture apparatus 101 as software. The software stored in the ROM 202 is deployed to the RAM 203, executed by the CPU 201, and realized by the CPU 201 controlling the units of the image capture apparatus 101.

When the power source button 401 of the image capture apparatus 101 is pressed and the power source enters an on-state, the processing of this flowchart is started by the CPU 201.

In step S8001, the CPU 201 displays the photographing person authentication screen in FIG. 5A on the display 408, and advances the procedure to step S8002.

In step S8002, the CPU 201 waits for a photographing person to input a medical worker ID and a password to the photographing person authentication screen in FIG. 5A and press the photographing person authentication button 503. When the photographing person authentication button 503 is pressed (step S713 in FIG. 7A), the CPU 201 advances the procedure to step S8003. Otherwise the CPU 201 returns the procedure to step S8002, and performs loop processing.

In step S8003, the CPU 201 transmits an authentication request to the image capture apparatus control apparatus 102 (step S714 in FIG. 7A). The authentication request includes the medical worker ID and the password input by the photographing person. Upon receiving the authentication response from the image capture apparatus control apparatus 102 (step S718 in FIG. 7A), the CPU 201 advances the procedure to step S8004.

In step S8004, the CPU 201 confirms a result of an authentication response. The CPU 201 advances the procedure to step S8005 if the content of the authentication response indicates that authentication was successfully performed, and returns the procedure to step S8002 if authentication failed.

In step S8005, the CPU 201 transmits a request to obtain patient information, to the image capture apparatus control apparatus 102 (step S719 in FIG. 7B). The request to obtain patient information includes the medical worker ID. Upon receiving a patient information obtaining response from the image capture apparatus control apparatus 102 (step S723 in FIG. 7B), the CPU 201 advances the procedure to step S8006.

In step S8006, the CPU 201 extracts the patient ID 601, the name (Chinese character) 602, the age 603, the gender 604 included in the patient information obtaining response, as patient information, stores the patient information in the RAM 203 (step S724 in FIG. 7B), and then displays the patient information display screen in FIG. 5B, on the display 408, and advances the procedure to step S8007. In the example in FIG. 5B, patient information is displayed in which the patient ID 601 is “0001”, the name (Chinese character) 602 is “Shimomaruko Hanako”, the age 603 is “35”, and the gender 604 is “female”.

In step S8007, the CPU 201 performs loop processing, and waits for the photographing person to press the release button 402, which is an example of an image capture instruction. When the release button 402 is pressed (step S725 in FIG. 7B), the CPU 201 advances the procedure to step S8008, otherwise the CPU 201 returns the procedure to step S8007.

In step S8008, the CPU 201 reads out a captured image obtained by pressing the release button 402, from the RAM 203. The CPU 201 then causes the file generation unit 211 to generate a lesion image in which the patient ID 601, the name (Chinese character) 602, the age 603, and the gender 604 stored in the RAM 203 in step S8006 are provided to the captured image as EXIF information, and stores the lesion image in the RAM 203 (step S727 in FIG. 7B). The CPU 201 then displays the lesion image display screen in FIG. 5C on the display 408, and advances the procedure to step S8009.

In step S8009, the CPU 201 performs loop processing, and waits for the photographing person to press one of the OK button 523, a cancel button 524, and the end button 525. When any one of the buttons is pressed, the CPU 201 advances the procedure to step S8010.

In step S8010, the CPU 201 advances the procedure to step S8013 if the end button 525 has been pressed, which indicates that lesion image capturing has ended (step S728 in FIG. 7B), otherwise the CPU 201 advances the procedure to step S8011.

In step S8011, the CPU 201 returns the procedure to step S8007 if the OK button 523 has been pressed, and advances the procedure to step S8012 if the OK button 523 has not been pressed and the cancel button 524 has been pressed.

In step S8012, the CPU 201 deletes the lesion image generated in step S8008 from the RAM 203. When deletion is complete, the CPU 201 returns the procedure to step S8007.

In step S8013, the CPU 201 transmits an image storing request to the image capture apparatus control apparatus 102 (step S729 in FIG. 7B). The image storing request includes the lesion image stored in the RAM 203 in step S8008. Upon receiving the image storing response from the image capture apparatus control apparatus 102 (step S731 in FIG. 7B), the CPU 201 advances the procedure to step S8014.

In step S8014, the CPU 201 advances the procedure to step S8015 if the image storing response indicates that the image was successfully stored, otherwise the CPU 201 returns the procedure to step S8013, and transmits an image storing request to the image capture apparatus control apparatus 102 again.

In step S8015, the CPU 201 deletes the lesion image generated in step S8008 from the RAM 203 (step S732 in FIG. 7B), and advances the procedure to step S8016.

In step S8016, the CPU 201 deletes the patient ID 601, the name (Chinese character) 602, the age 603, and the gender 604 stored in step S8006, from the RAM 203 (step S733 in FIG. 7B), and ends the processing.

Description will be given with reference to FIG. 8B. Processing of the flowchart in FIG. 8B is stored in the ROM 302 of the image capture apparatus control apparatus 102 as software. The software stored in the ROM 302 is deployed to the RAM 303, executed by the CPU 301, and realized by the CPU 301 controlling the units of the image capture apparatus control apparatus 102.

When the power source button (not illustrated) of the image capture apparatus control apparatus 102 is pressed and the power source enters an on-state, the processing of this flowchart is started by the CPU 301.

In step S8101, when the power source button (not illustrated) of the image capture apparatus control apparatus 102 is pressed and the power source enters an off-state, the CPU 301 ends the processing. Otherwise the CPU 301 advances the procedure to step S8102.

In step S8102, the CPU 301 determines whether or not any of an authentication request from the image capture apparatus 101 (step S714 in FIG. 7A), a request to obtain patient information (step S719 in FIG. 7B), and an image storing request (step S729 in FIG. 7B) has been received. If any of the requests has been received, the CPU 301 advances the procedure to step S8103, otherwise the CPU 301 returns the procedure to step S8101, and performs loop processing.

In step S8103, if an authentication request has been received (step S714 in FIG. 7A), the CPU 301 advances the procedure to step S8104. Otherwise (if a request to obtain patient information or an image storing request has been received), the CPU 301 advances the procedure to step S8106.

In step S8104, the CPU 301 transmits the authentication request received from the image capture apparatus 101, to the electronic medical record management apparatus 104 (step S715 in FIG. 7A), and advances the procedure to step S8105.

In step S8105, upon receiving an authentication response from the electronic medical record management apparatus 104 (step S717 in FIG. 7A), the CPU 301 transmits the authentication response to the image capture apparatus 101 (step S718 in FIG. 7A), and returns the procedure to step S8101.

In step S8106, if a request to obtain patient information has been received (step S719 in FIG. 7B), the CPU 301 advances the procedure to step S8107. Otherwise (if an image storing request has been received), the CPU 301 advances the procedure to step S8109.

In step S8107, the CPU 301 transmits the request to obtain patient information, which has been received from the image capture apparatus 101, to the electronic medical record management apparatus 104 (step S721 in FIG. 7B), and advances the procedure to step S8108.

In step S8108, upon receiving a patient information obtaining response from the electronic medical record management apparatus 104 (step S722 in FIG. 7B), the CPU 301 transmits an authentication response to the image capture apparatus 101 (step S723 in FIG. 7B), and returns the procedure to step S8101.

In step S8109, the CPU 301 obtains the lesion image included in the image storing request received from the image capture apparatus 101, and stores the image in the image management apparatus 105. If the lesion image is successfully stored in the image management apparatus 105, the CPU 301 transmits an image storing response indicating that the image was successfully stored. Otherwise the CPU 301 transmits an image storing response indicating that storing the image failed, and returns the procedure to step S8101.

Description will be given with reference to FIG. 8C. Processing of the flowchart in FIG. 8C is stored in the ROM 302 of the electronic medical record terminal apparatus 103 as software. The software stored in the ROM 302 is deployed to the RAM 303, executed by the CPU 301, and realized by the CPU 301 controlling the units of the electronic medical record terminal apparatus 103.

When the power source button (not illustrated) of the electronic medical record terminal apparatus 103 is pressed and the power source enters an on-state, the processing of this flowchart is started by the CPU 301.

In step S8201, the CPU 301 waits for a medical practitioner that is a medical worker to input a medical worker ID and a password. When a medical worker ID and a password are input, the CPU 301 advances the procedure to step S8202, otherwise the CPU 301 returns the procedure to step S8201, and performs loop processing.

In step S8202, the CPU 301 transmits an authentication request to the electronic medical record management apparatus 104 (step S702 in FIG. 7A). The authentication request includes the medical worker ID and the password input by the medical practitioner, who is a medical worker. Upon receiving the authentication response from the electronic medical record management apparatus 104 (step S704 in FIG. 7A), the CPU 301 advances the procedure to step S8203.

In step S8203, the CPU 301 confirms the result of the authentication response. The CPU 301 advances the procedure to step S8204 if authentication was successfully performed, and returns the procedure to step S8201 if authentication failed.

In step S8204, the CPU 301 waits for the medical practitioner, who is a medical worker, to input a patient ID. When a patient ID is input, the CPU 301 advances the procedure to step S8205. Otherwise the CPU 301 returns the procedure to step S8204, and performs loop processing.

In step S8205, the CPU 301 transmits a request to obtain medical record information, to the electronic medical record management apparatus 104 (step S707 in FIG. 7A). The request to obtain medical record information includes a medical worker ID and a patient ID. Upon receiving a medical record information obtaining response, from the electronic medical record management apparatus 104 (step S710 in FIG. 7A), the CPU 301 advances the procedure to step S8206.

In step S8206, the CPU 301 obtains medical record information from the medical record information obtaining response, displays the medical record information on the display unit 305 of the electronic medical record terminal apparatus 103, and advances the procedure to step S8207.

In step S8207, the CPU 301 performs loop processing, and waits for the medical practitioner, who is a medical worker, to perform input for performing clinical examination on the next patient or input for logging off to end the medical examination operation. Upon receiving input from the medical practitioner, who is a medical worker, the CPU 301 advances the procedure to step S8208, otherwise the CPU 301 returns the procedure to step S8207.

In step S8208, the CPU 301 determines whether or not input for logging off has been accepted. If input for logging off has been accepted (step S734 in FIG. 7B), the procedure advances to step S8209. Otherwise the CPU 301 returns the procedure to step S8204.

In step S8209, the CPU 301 transmits a log-off request to the electronic medical record management apparatus 104 (step S735 in FIG. 7B). Upon receiving the log-off response from the electronic medical record management apparatus 104 (step S738 in FIG. 7B), the CPU 301 ends the processing.

Description will be given with reference to FIG. 8D. Processing of the flowchart in FIG. 8D is stored in the ROM 302 of the electronic medical record management apparatus 104 as software. The software stored in the ROM 302 is deployed to the RAM 303, executed by the CPU 301, and realized by the CPU 301 controlling the units of the electronic medical record management apparatus 104.

When the power source button (not illustrated) of the electronic medical record management apparatus 104 is pressed and the power source enters an on-state, the processing of this flowchart is started by the CPU 301.

In step S8301, when the power source button (not illustrated) of the electronic medical record management apparatus 104 is pressed and the power source enters an on-state, the CPU 301 ends the processing. Otherwise the CPU 301 advances the procedure to step S8302.

In step S8302, the CPU 301 determines whether or not one of an authentication request from the electronic medical record terminal apparatus 103 (step S703 in FIG. 7A), a request to obtain medical record information from the electronic medical record terminal apparatus 103 (step S707 in FIG. 7A), an authentication request from the image capture apparatus control apparatus 102 (step S715 in FIG. 7A), a request to obtain patient information from the image capture apparatus control apparatus 102 (step S720 in FIG. 7B), and a log-off request from the electronic medical record terminal apparatus 103 (step S735 in FIG. 7B) has been received. If any of the requests has been received, the CPU 301 advances the procedure to step S8303, otherwise the CPU 301 returns the procedure to step S8301, and performs loop processing.

In step S8303, if an authentication request has been received (step S702 or S715 in FIG. 7A), the CPU 301 advances the procedure to step S8304, otherwise the CPU 301 advances the procedure to step S8306.

In step S8304, the CPU 301 determines whether or not the medical worker ID and the password included in the received authentication request match a combination of a medical worker ID 611 and a password 613 in the medical worker information table in FIG. 6B, and advances the procedure to step S8305.

In step S8305, if the medical worker ID and the password included in the received authentication request match a medical worker ID 611 and a password 613 in the medical worker information table, the CPU 301 transmits an authentication response indicating that authentication was successfully performed (step S704 or S717 in FIG. 7A). If the medical worker IDs and the passwords do not match, the CPU 301 transmits an authentication response indicating that authentication failed (step S704 or S717 in FIG. 7A). Then, when processing for transmitting the authentication response ends, the CPU 301 returns the procedure to step S8301.

In step S8306, if a request to obtain medical record information has been received (step S707 in FIG. 7A), the CPU 301 advances the procedure to step S8307, otherwise the CPU 301 advances the procedure to step S8310.

In step S8307, the CPU 301 obtains the patient information table (FIG. 6A) and the patient clinical examination information table (not illustrated) stored in the HDD 308 of the electronic medical record management apparatus 104, generates medical record information, and advances the procedure to step S8308.

In step S8308, the CPU 301 updates the clinical examination patient information table (FIG. 6C) stored in the HDD 308 of the electronic medical record management apparatus 104. Specifically, the CPU 301 obtains the medical worker ID and the patient ID from the request to obtain medical record information, and registers the combination of the medical worker ID and the patient ID to the clinical examination patient information table. If the medical worker ID has already been registered in the clinical examination patient information table, the CPU 301 updates a patient ID corresponding to the registered medical worker ID. When processing for updating the clinical examination patient information table (FIG. 6C) ends, the CPU 301 advances the procedure to step S8309.

In step S8309, the CPU 301 adds the medical record information generated in step S8307 to a medical record information obtaining response, transmits the medical record information obtaining response (step S710 in FIG. 7A), and, when transmission is complete, returns the procedure to step S8301.

In step S8310, if a request to obtain patient information (step S720 in FIG. 7B) has been received, the CPU 301 advances the procedure to step S8311. Otherwise (if a log-off request (step S735 in FIG. 7B) has been received) the CPU 301 advances the procedure to step S8313.

In step S8311, the CPU 301 obtains the medical worker ID from the request to obtain patient information. The CPU 301 obtains the patient ID associated with the obtained medical worker ID, from the clinical examination patient information table (FIG. 6C) stored in the HDD 308 of the electronic medical record management apparatus 104. The CPU 301 then obtains patient information (the patient ID 601, the name (Chinese character) 602, the age 603, and the gender 604) of the patient ID obtained from the patient information table (FIG. 6A) stored in the HDD 308 of the electronic medical record management apparatus 104, and advances the procedure to step S8312.

In step S8312, the CPU 301 adds the patient information obtained in step S8311, to a patient information obtaining response, transmits the patient information obtaining response (step S723 in FIG. 7B), and, when transmission is complete, returns the procedure to step S8301.

In step S8313, the CPU 301 obtains the medical worker ID included in the log-off request, deletes the record of the medical worker ID from the clinical examination patient information table (FIG. 6C) stored in the HDD 308 of the electronic medical record management apparatus 104, and advances the procedure to step S8314.

In step S8314, the CPU 301 transmits a log-off response (step S737 in FIG. 7B). When processing for transmitting the log-off response ends, the procedure returns to step S8301.

As described above, the patient ID of a patient whose medical record information is displayed (viewed) on the electronic medical record terminal apparatus 103 that is being operated by a medical worker (that a medical worker has logged into) is associated with the medical worker that is operating the electronic medical record terminal apparatus 103, and the patient ID and the medical worker are managed as a clinical examination patient information table. In addition, from the clinical examination patient information table, the patient ID of the patient that is displayed (viewed) on the electronic medical record terminal apparatus that a medical worker, which is the same as a photographing person of the image capture apparatus 101 and a specified medical worker, is operating (has logged into) is specified, and patient information of the specified patient ID is transmitted to the image capture apparatus. The image capture apparatus 101 transmits a lesion image and a patient image as a set to the image capture apparatus control apparatus 102 of a management system, and thus it is possible to easily associate the lesion image shot by the medical worker and patient information of the patient with each other.

The electronic medical record management apparatus 104 associates the medical worker ID of a medical practitioner and the patient ID of a patient under clinical examination with each other, and thus, simply by an authentication request being made from the image capture apparatus 101 by a medical practitioner viewing the electronic medical records of a patient who is to be subjected to lesion image capturing, patient information can be set in the authenticated image capture apparatus 101. For this reason, it is possible to associate patient information of a patient for which a photographing person performs lesion image capturing and a captured image of a lesion with each other.

By a medical worker pressing the release button, which is equivalent to an instruction to capture an image, the image capture apparatus 101 generates a lesion image obtained by capturing an image of a lesion of a patient, and associates patient information with the lesion image. Accordingly, the medical worker can associate the patient information with the lesion image without performing a separate operation.

A preferred embodiment of the present invention have been described, but the present invention is not limited to the embodiment, and various changes and modifications can be made without departing from the spirit of the present invention.

In the embodiment, an example has been described in which authentication of a medical worker in the image capture apparatus 101 is performed by the electronic medical record management apparatus 104, but a configuration may also be adopted in which information of a medical worker information table is stored in the image capture apparatus 101, and the image capture apparatus 101 performs authentication of a medical worker.

In the embodiment, an example has been described in which authentication of a medical worker in the image capture apparatus 101 is password authentication, but any form of authentication may be used as long as authentication can be performed. Besides memory-based authentication such as password authentication, possession-based authentication in which an IC card is used, or biometric authentication such as fingerprint authentication, face authentication, or iris authentication may also be used as authentication of a medical worker. In addition, multi-factor authentication in which a plurality of types of authentications such as memory-based authentication, possession-based authentication, and biometric authentication are combined may also be used as authentication of a medical worker.

In the embodiment, a description has been given assuming that the medical worker is a medical practitioner, but the medical worker may be a medical worker that shoots an image of a lesion of a lesion, such as a clinical nurse or a radiation technologist.

In addition, the present embodiment will be supplemented.

For simplification, the present embodiment has been described assuming that a configuration is adopted in which there are one image capture apparatus 101 and one electronic medical record terminal apparatus 103, but there may be a plurality of image capture apparatuses 101 and a plurality of electronic medical record terminal apparatuses 103.

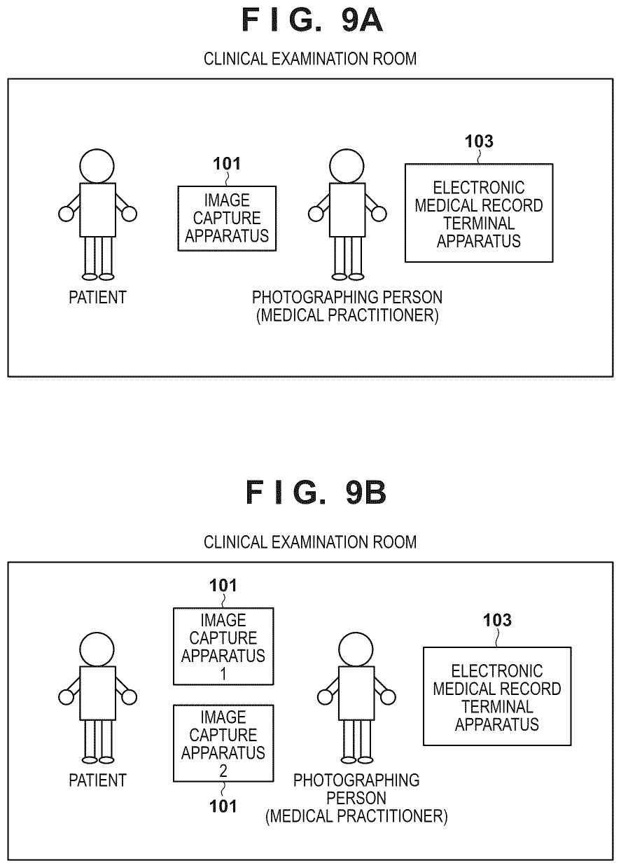

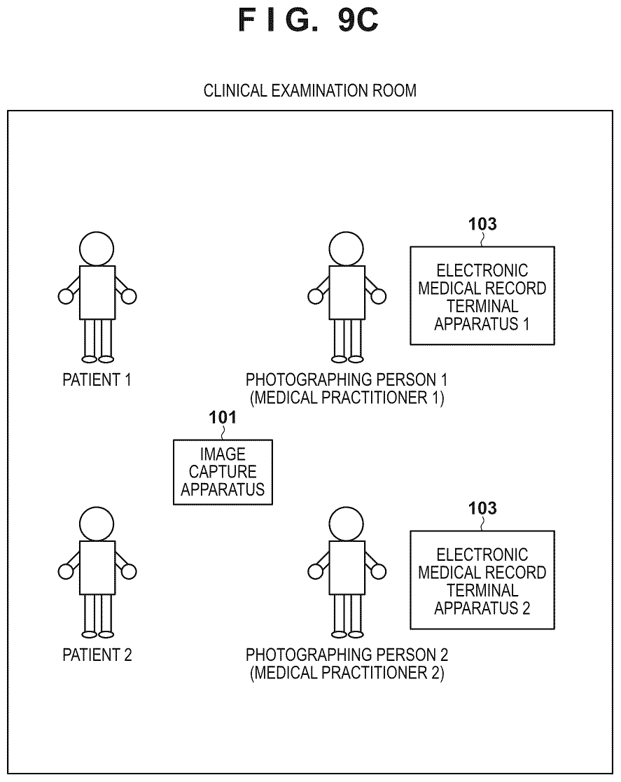

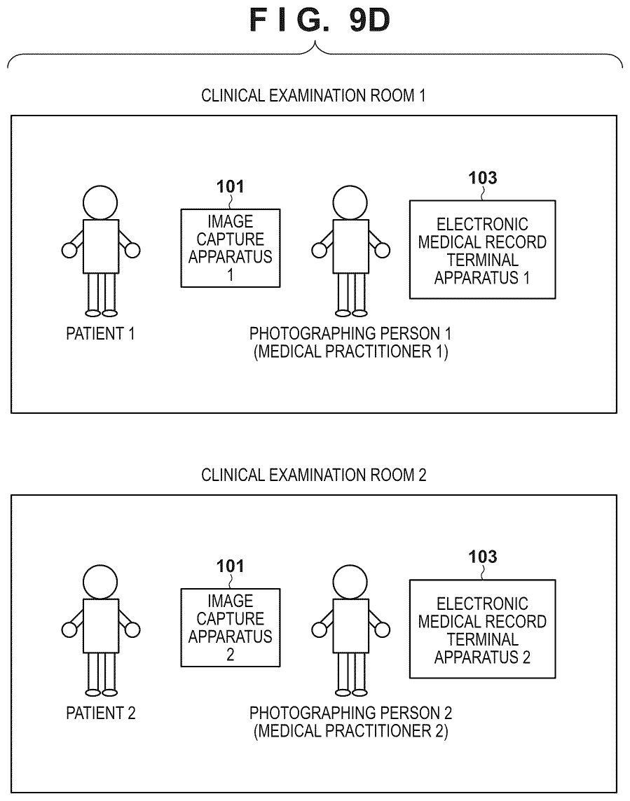

A configuration in which there are one image capture apparatus 101 and one electronic medical record terminal apparatus 103, which has been described in the present embodiment, specifically envisions operations in a clinic with one medical practitioner. In a configuration in a case where one image capture apparatus 101 is used for capturing an image of a lesion, the operations in FIG. 9A are adopted. In addition, in a configuration in a case where a dermo camera that is used for taking a close-up image of skin is also used as the image capture apparatus 101, the operations in FIG. 9B are adopted. Even in operations such as those illustrated in FIG. 9B, patient information of a patient on which medical practitioner is performing clinical examination can be set in any image capture apparatus 101, and thus the present embodiment is effective.

In addition, FIG. 9C shows an operation example in which two medical practitioners share and use one image capture apparatus 101. In FIG. 9C, each medical practitioner that needs to shoot an image of a lesion picks up the image capture apparatus 101 and shoots an image of a lesion of his or her patient. Even in operations such as those illustrated in FIG. 9C, patient information of a patient on which a medical practitioner is performing clinical examination can be set in the image capture apparatus 101, and thus the present embodiment is effective. In addition, there is an effect that, even when a plurality of patients are under clinical examination at the same time, patient information of the patients on which medical practitioners are performing clinical examination can be appropriately set in the only one image capture apparatus 101.

In addition, FIG. 9D envisions operations in a large-scale hospital in which medical practitioners are present respectively in a plurality of clinical examination rooms and perform clinical examination on patients. In such a case, the image capture apparatus 101 and the electronic medical record terminal apparatus 103 are disposed in each of the clinical examination rooms. Also in a case of operations such as those illustrated in FIG. 9D, in any image capture apparatus 101, patient information of a patient on which the medical practitioner is performing clinical examination can be set, and thus the present embodiment is effective. In addition, there is an effect that, even when a plurality of patients are under clinical examination at the same time, patient information of a patient on which a medical practitioner is performing clinical examination can be appropriately set in the image capture apparatus 101 that is capturing an image of a lesion of the patient from among the plurality of image capture apparatuses 101.

A configuration may also be adopted in which, when a medical practitioner logs into the electronic medical record terminal apparatus 103, requests for medical record information of a patient who is to be subjected to clinical examination, from the electronic medical record management apparatus 104, and obtains the medical record information, medical record content of this patient is displayed on a screen of the electronic medical record terminal apparatus 103, and an select item for selecting “capture an image” or “do not capture an image” is also displayed. When “capture an image” is selected, the electronic medical record terminal apparatus 103 transmits information indicating that “capture an image” has been selected, to the electronic medical record management apparatus 104. When receiving of the information is detected, the electronic medical record management apparatus 104 holds the medical practitioner ID of the medical practitioner who performed the operation, the patient ID, and information indicating that an image is to be captured, and waits for the medical practitioner to log in from the image capture apparatus 101. When the medical practitioner logs in from the image capture apparatus 101, the electronic medical record management apparatus 104 performs authentication processing. After that, when authentication was successfully performed, and a user ID input at the time of log-in from the image capture apparatus 101 matches the previously held medical practitioner ID, the electronic medical record management apparatus 104 may transmit information regarding the patient (patient ID, name, gender, age, image capture segment, etc.) to the image capture apparatus 101 that has logged in. As a result, when logging in from the image capture apparatus 101, the medical practitioner that is operating the image capture apparatus 101 can immediately confirm whether or not there is an error in the patient whose image is to be captured, and the like.

### Second Embodiment

A second embodiment is obtained by extending the first embodiment. In the second embodiment, a description will be given on not only a case in which a medical worker logs into the electronic medical record terminal apparatus 103, and performs clinical examination on a patient, but also an example in which an image of a patient ID that is an ID for specifying a patient is captured, and the image capture apparatus 101 associates patient information and a lesion image with each other.

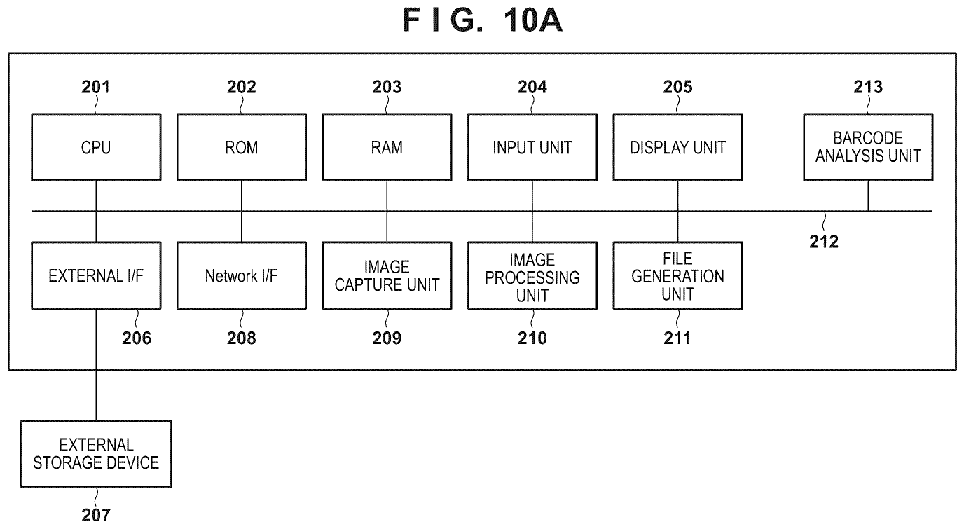

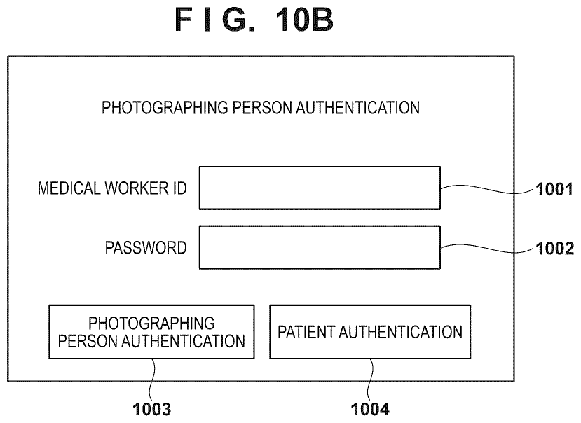

FIG. 10A is a block diagram of an image capture apparatus according to the second embodiment. In the second embodiment, the image capture apparatus 101 also includes a barcode analysis unit 213 connected to the CPU bus 212. The barcode analysis unit 213 analyzes an image file that includes a barcode image in a general-purpose still image format generated by the file generation unit 211, and obtains barcode character strings. The CPU 201 causes the barcode analysis unit 213 to perform barcode analysis on an image file obtained by the file generation unit 211 converting image data generated by the image processing unit 210, into a general-purpose still image format. If analysis is successfully performed, the barcode analysis unit 213 returns barcode character strings. In the second embodiment, barcode analysis is performed on a barcode image of a patient ID, and thus, if analysis is successfully performed, the patient ID is returned. A barcode image is also referred to as “identification information image”. Note that the barcode analysis unit 213 may be realized as a function of the CPU 201 that executes software for analyzing a barcode.

FIG. 10B is a photographing person authentication screen that is displayed on the display 408 of the image capture apparatus 101.

A medical worker ID input area 1001 is similar to the medical worker ID input area 501, and thus a description thereof is omitted. A password input area 1002 is similar to the password input area 502, and thus a description thereof is omitted. A photographing person authentication button 1003 is similar to the photographing person authentication button 503, and thus a description thereof is omitted. A patient authentication button 1004 is a button that is pressed when an image of patient ID barcode is captured and patient information is obtained.

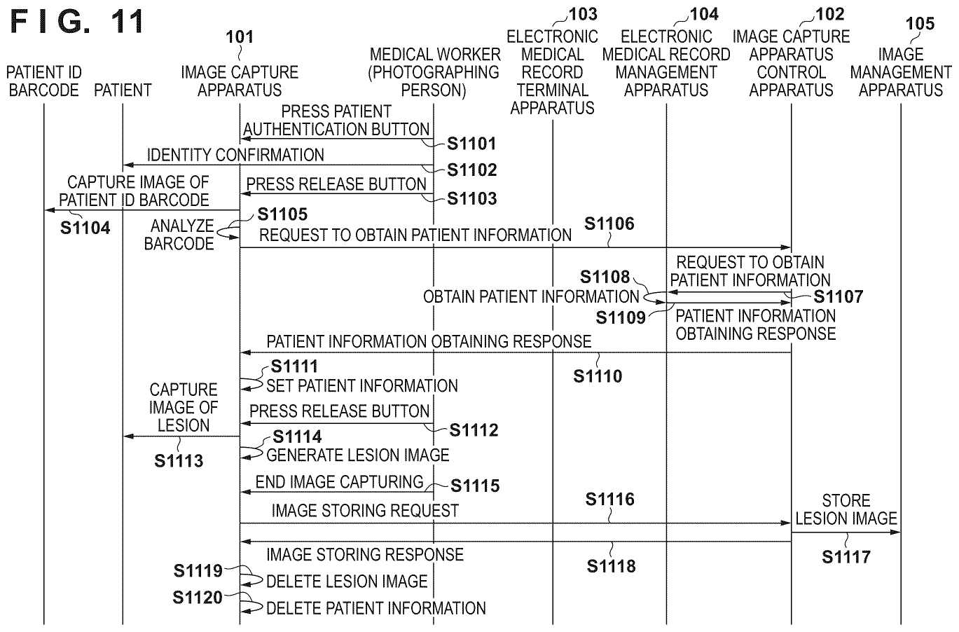

FIG. 11 is a diagram of a processing sequence when an image of a patient ID barcode is captured in an image capture system. In the second embodiment, the photographing person authentication screen in FIG. 10B is displayed on the display 408 of the image capture apparatus 101.

In the second embodiment, when a medical practitioner inputs a medical worker ID and a password on the photographing person authentication screen in FIG. 10B, and presses the photographing person authentication button 1003, step S713 is started. When the medical practitioner presses the patient authentication button 1004, step S1101 in FIG. 11 is started.

In step S1102, the medical practitioner performs identity confirmation, that is, confirms that the patient is a patient who is to be subjected to lesion image capturing.

In step S1103, the medical practitioner turns the image capture apparatus 101 towards the patient ID barcode, and presses the release button 402 of the image capture apparatus 101. Note that the patient ID barcode is obtained by bar-coding the patient ID 601 of the patient and printing the bar-coded ID onto a sheet, which is carried by the patient when he is she is in the hospital.

In step S1104, the image capture apparatus 101 captures an image of the patient ID barcode, and generates barcode image data.

In step S1105, the image capture apparatus 101 performs processing for analyzing the image data of the patient ID barcode generated in step S1104, and obtaining a patient ID. In the present embodiment, barcode image data is converted into a JPEG image, and the JPEG image is analyzed by the barcode analysis unit 213, and a patient ID is obtained.

In step S1106, the image capture apparatus 101 transmits, to the image capture apparatus control apparatus 102, a request to obtain patient information corresponding to the patient ID obtained in step S1105.

In step S1107, the image capture apparatus control apparatus 102 transmits the received request to obtain patient information, to the electronic medical record management apparatus 104.

In step S1108, the electronic medical record management apparatus 104 performs processing for obtaining patient information, in accordance with the received request to obtain patient information. In processing for obtaining patient information, the electronic medical record management apparatus 104 obtains the patient ID included in the request to obtain patient information, and obtains patient information corresponding to the patient ID from the patient information table in FIG. 6A.

In step S1109, the electronic medical record management apparatus 104 transmits the patient information obtained in step S1108 to the image capture apparatus control apparatus 102 as a patient information obtaining response.

In step S1110, the image capture apparatus control apparatus 102 transmits the received patient information obtaining response to the image capture apparatus 101.

Steps S1111 to S1120 are similar to steps S724 to S733, and thus a description thereof is omitted.

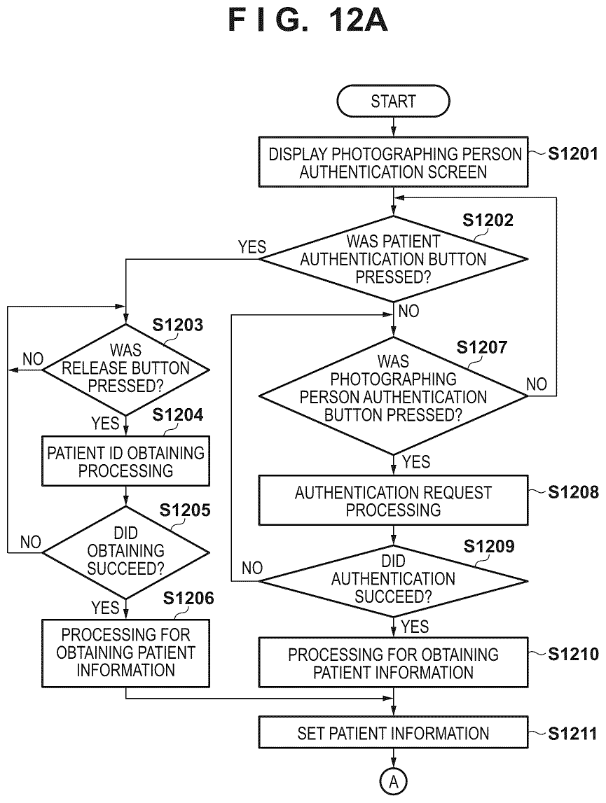

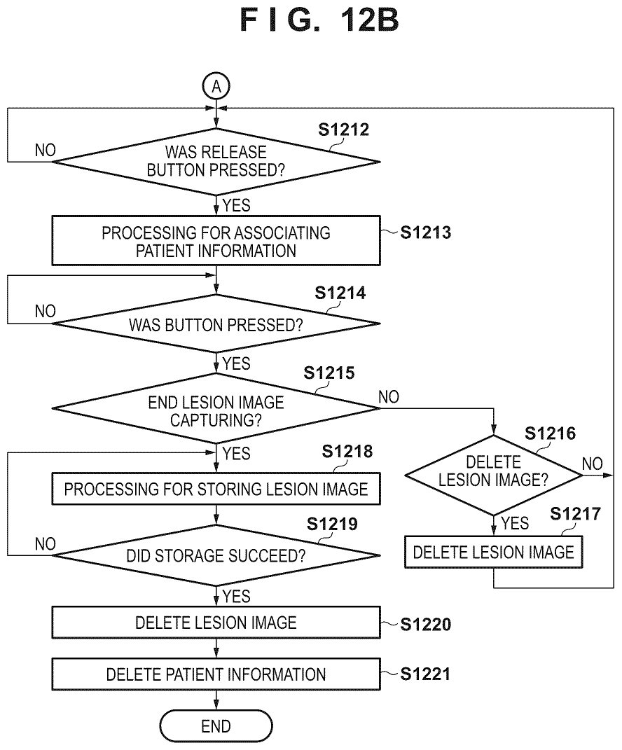

FIGS. 12A and 12B is a flowchart of the image capture apparatus 101 according to the second embodiment.

Processing of the flowchart in FIGS. 12A and 12B is stored in the ROM 202 of the image capture apparatus 101 as software. The software stored in the ROM 202 is deployed to the RAM 203, executed by the CPU 201, and realized by the CPU 201 controlling the units of the image capture apparatus 101.

When the power source button 401 of the image capture apparatus 101 is pressed and the power source enters an on-state, the processing of this flowchart is started by the CPU 201.

In step S1201, the CPU 201 displays the photographing person authentication screen in FIG. 10B on the display 408, and advances the procedure to step S1202.

In step S1202, the CPU 201 confirms whether or not a photographing person has pressed the patient authentication button 1004 on the photographing person authentication screen (FIG. 10B). If the patient authentication button 1004 has been pressed (step S1101 in FIG. 11), the CPU 201 advances the procedure to step S1203, otherwise the CPU 201 advances the procedure to step S1207.

In step S1203, the CPU 201 performs loop processing, and waits for the photographing person to press the release button 402. When the release button 402 is pressed (step S1103 in FIG. 11), the CPU 201 advances the procedure to step S1204, otherwise the CPU 201 returns the procedure to step S1203.

In step S1204, the CPU 201 performs processing for analyzing image data of a captured image of a patient ID barcode and obtaining a patient ID, and advances the procedure to step S1205.

In step S1205, when the CPU 201 performs barcode analysis and successfully obtains a patient ID, the CPU 201 advances the procedure to step S1206, otherwise the CPU 201 returns the procedure to step S1203.

In step S1206, the CPU 201 transmits a request to obtain patient information, to the image capture apparatus control apparatus 102 (step S1106 in FIG. 11). The request to obtain patient information includes the patient ID. Upon receiving a patient information obtaining response from the image capture apparatus control apparatus 102 (step S1110 in FIG. 11), the CPU 201 advances the procedure to step S1212.

In step S1207, the CPU 201 confirms whether or not the photographing person has pressed the photographing person authentication button 1003 on the photographing person authentication screen (FIG. 10B). When the photographing person authentication button 1003 is pressed (step S713 in FIG. 7), the CPU 201 advances the procedure to step S1208, otherwise the CPU 201 returns the procedure to step S1202.

Steps S1208 to S1221 are similar to steps S8003 to S8016, and thus a description thereof is omitted.

The flowchart of processing of the image capture apparatus control apparatus 102 according to the second embodiment is similar to that of the first embodiment, and thus a description thereof is omitted.

The flowchart of processing of the electronic medical record terminal apparatus 103 according to the second embodiment is similar to that of the first embodiment, and thus a description thereof is omitted.

The flowchart of processing of the electronic medical record management apparatus 104 according to the second embodiment is substantially the same as that of the first embodiment. In the second embodiment, the processing of step S8311 is changed as follows.

In step S8311, if a request to obtain patient information includes a medical worker ID, the CPU 301 of the electronic medical record management apparatus 104 obtains the medical worker ID, and obtains a patient ID from the clinical examination patient information table (FIG. 6C) stored in the HDD 308 of the electronic medical record management apparatus 104. The CPU 301 then obtains patient information (the patient ID 601, the name (Chinese character) 602, the age 603, and the gender 604) of the patient ID obtained from the patient information table (FIG. 6A) stored in the HDD 308 of the electronic medical record management apparatus 104, and advances the procedure to step S8312. If the request to obtain patient information includes a patient ID, the CPU 301 obtains the patient ID, obtains patient information (the patient ID 601, the name (Chinese character) 602, the age 603, and the gender 604) of the patient ID obtained from the patient information table (FIG. 6A) stored in the HDD 308 of the electronic medical record management apparatus 104, and advances the procedure to step S8312.

Processing other than processing of step S8311 is similar to that of the first embodiment, and thus a description thereof is omitted.

As described above, not only in a case where a medical worker logs into the electronic medical record terminal apparatus 103 and performs clinical examination on a patient, but also by capturing an image of a barcode of a patient ID for specifying a patient, the image capture apparatus 101 can associate patient information and a lesion image with each other.

Using the image capture apparatus 101 according to the second embodiment, patient information and a lesion image can be easily associated with each other when a medical worker carries the image capture apparatus 101. Specifically, when the medical record of a patient is displayed on an electronic medical record terminal apparatus in a treatment room or the like, the image capture apparatus 101 obtains patient information using medical worker authentication, as in the first embodiment. When bedsores (a pressure ulcer) of a patient are found in an inpatients ward, and an image of the lesion is immediately captured, for example, as in the second embodiment, an image of the patient ID that the patient is carrying is captured, and the image capture apparatus 101 obtains patient information. Such a configuration has an effect that a medical worker can associate the patient information and the lesion image with each other.

### Third Embodiment

A third embodiment is obtained by extending the first embodiment. In the third embodiment, when photographing person authentication in step S714 according to the first embodiment fails due to a communication failure, and patient information cannot be obtained before lesion image capturing, patient information and a captured image are associated with each other after lesion image capturing is completed.

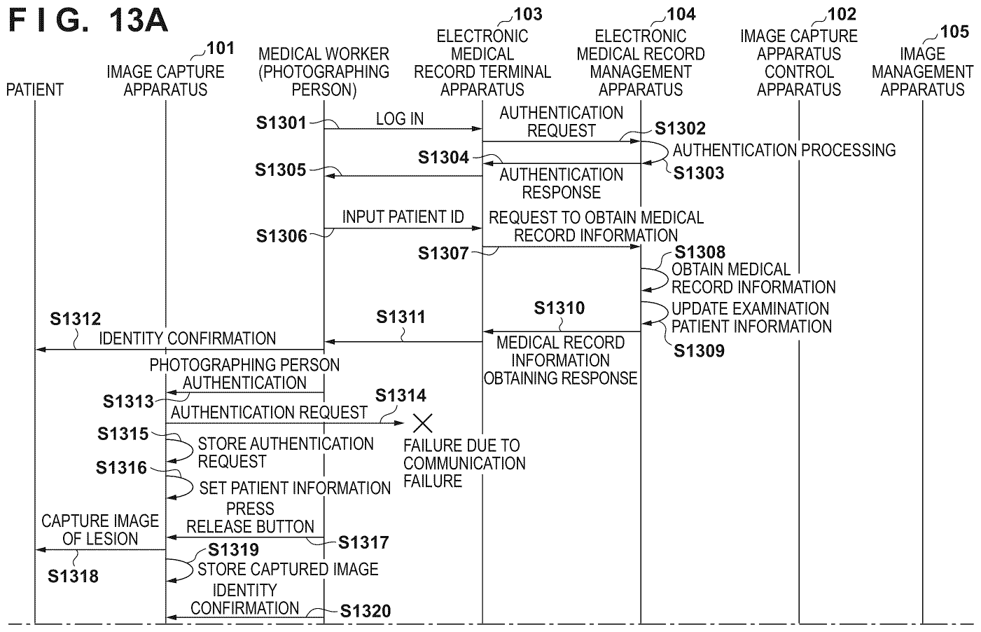

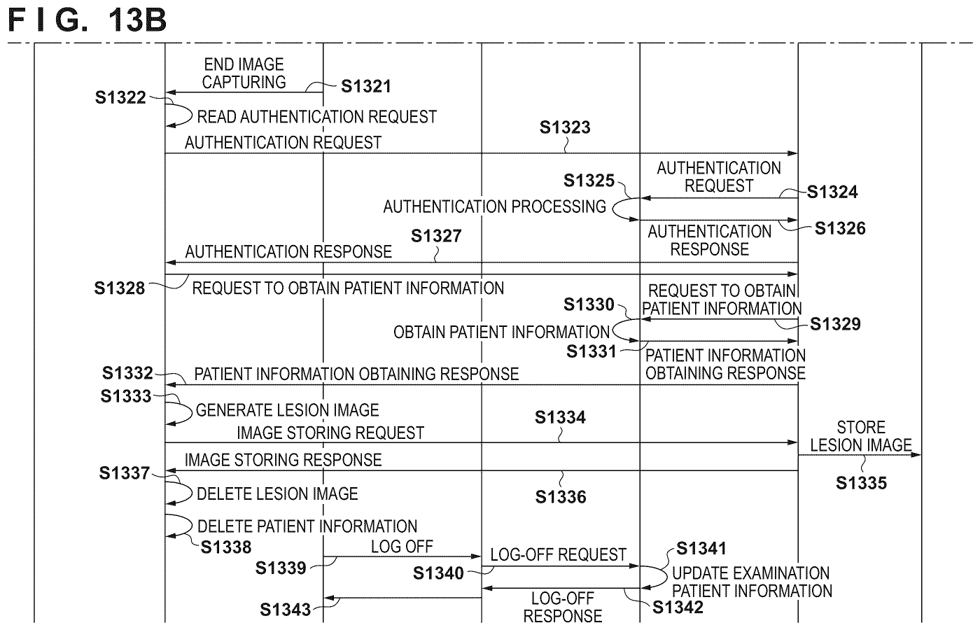

FIGS. 13A and 13B is a diagram of a processing sequence of an image capture system. Steps S1301 to S1313 are similar to steps S701 to S713, and thus a description thereof is omitted.

In step S1314, the image capture apparatus 101 transmits, to the image capture apparatus control apparatus 102, a request to authenticate a medical worker ID and a password input by a medical practitioner, but the transmission fails due to a communication failure. If an authentication response to the authentication request is not received from the image capture apparatus control apparatus 102 after the image capture apparatus 101 has waited for a predetermined time, for example, it may be determined that a communication failure has occurred.

In step S1315, the image capture apparatus 101 stores, in the RAM 203, the authentication request transmitted in step S1314.

Steps S1316 to S1318 are similar to steps S724 to S726, and thus a description thereof is omitted.

In step S1319, the image capture apparatus 101 converts the image data captured in step S1318 into a general-purpose still image format, and generates an image file. In the present embodiment, the image capture apparatus 101 converts lesion image data into a JPEG image.

In step S1320, the medical practitioner performs identity confirmation, that is, confirms that the electronic medical record information displayed on the electronic medical record terminal apparatus 103 in step S1311 is the electronic medical record information of the patient himself or herself.

In step S1321, the medical practitioner presses the end button 525 displayed on the display unit 205 of the image capture apparatus 101 in order to end lesion image capturing.

In step S1322, the image capture apparatus 101 reads the authentication request stored in the RAM 203 in step S1315 in order to make an authentication request in step S1323.

Steps S1323 to S1332 are similar to steps S714 to S723, and thus a description thereof is omitted.

In step S1333, the image capture apparatus 101 performs processing for associating the patient information obtained in step S1332 and the image file generated in step S1319 with each other. In the present embodiment, the image capture apparatus 101 sets the patient information (patient ID, patient name, and the like) in EXIF information of the image file.

Steps S1334 to S1343 are similar to steps S729 to S738, and thus a description thereof is omitted.

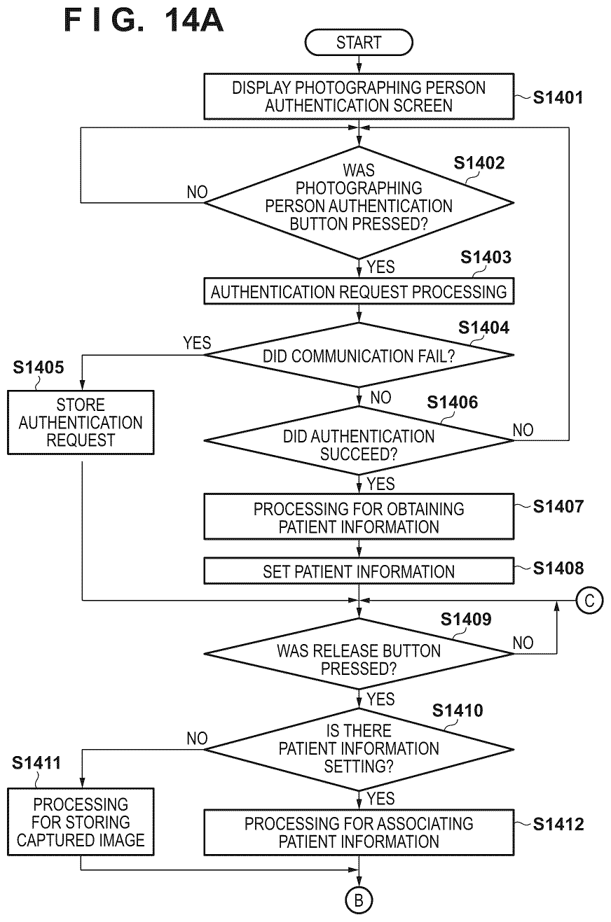

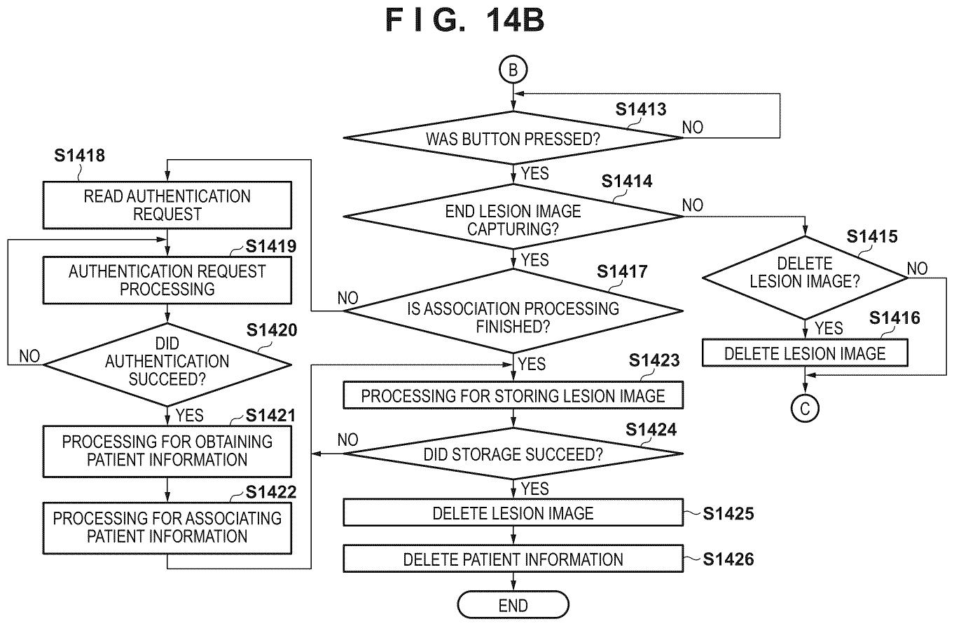

FIGS. 14A and 14B is a flowchart of the image capture apparatus 101 for realizing the processing sequence of the image capture system described with reference to FIGS. 13A and 13B.

Processing of the flowchart in FIGS. 14A and 14B is stored in the ROM 202 of the image capture apparatus 101 as software. The software stored in the ROM 202 is deployed to the RAM 203, executed by the CPU 201, and realized by the CPU 201 controlling the units of the image capture apparatus 101.

When the power source button 401 of the image capture apparatus 101 is pressed, and the power source enters an on-state, the processing of this flowchart is started by the CPU 201. Steps S1401 and S1402 are similar to steps S8001 and S8002, and thus a description thereof is omitted.

In step S1403, the CPU 201 transmits an authentication request to the image capture apparatus control apparatus 102 (step S714 in FIG. 7). The authentication request includes the medical worker ID and the password input by the photographing person. When an authentication response is received from the image capture apparatus control apparatus 102 (step S718 in FIG. 7), or when a communication failure such as communication timeout occurs (step S1314 in FIG. 13A), the CPU 201 advances the procedure to step S1404.

In step S1404, the CPU 201 confirms a result of the authentication response. In the case of a communication failure, the CPU 201 advances the procedure to step S1405, otherwise the CPU 201 advances the procedure to step S1406.

In step S1405, the CPU 201 stores the authentication request in step S1403 in the RAM 203, and advances the procedure to step S1409.

Steps S1406 to S1409 are similar to steps S8004 to S8007, and thus a description thereof is omitted.

In step S1410, the CPU 201 confirms whether or not patient information has been set in the RAM 203 in step S1408. The CPU 201 advances the procedure to step S1411 if patient information has not been set due to a communication failure or the like, and advances the procedure to step S1412 if patient information has been set as a result of authentication having been successfully performed.

In step S1411, the CPU 201 reads out captured image data obtained by pressing the release button 402, from the RAM 203. The CPU 201 then causes the file generation unit 211 to convert the captured image data into a general-purpose still image format, stores the data in the RAM 203 (step S1319 in FIG. 13A), and advances the procedure to step S1413.

Steps S1412 to S1416 are similar to steps S8008 to S8012, and thus a description thereof is omitted.

In step S1417, the CPU 201 examines whether or not patient information is associated with the image file stored in the RAM 203. The CPU 201 advances the procedure to step S1418 if patient information is not associated with the image file, and advances the procedure to step S1423 if patient information is associated with the image file.

In step S1418, the CPU 201 reads the authentication request stored in the RAM 203 in step S1405, and advances the procedure to step S1419.

In step S1419, the CPU 201 transmits an authentication request to the image capture apparatus control apparatus 102 (step S1323 in FIG. 13B). Upon receiving an authentication response from the image capture apparatus control apparatus 102 (step S1327 in FIG. 13B), the CPU 201 advances the procedure to step S1420.

In step S1420, the CPU 201 confirms a result of the authentication response. The CPU 201 advances the procedure to step S1421 if authentication was successfully performed, returns the procedure to step S1419 if authentication failed, and executes authentication request processing that includes transmission of an authentication request and the like, again.

In step S1421, the CPU 201 transmits a request to obtain patient information, to the image capture apparatus control apparatus 102 (step S1328 in FIG. 13B). The request to obtain patient information includes a medical worker ID. Upon receiving a patient information obtaining response from the image capture apparatus control apparatus 102 (step S1336 in FIG. 13B), the CPU 201 advances the procedure to step S1422.

In step S1422, the CPU 201 extracts the patient ID 601, the name (Chinese characters) 602, the age 603, and the gender 604 that are included in the patient information obtaining response, and stores the extracted information in the RAM 203. The CPU 201 then causes the file generation unit 211 to generates a lesion image by adding the patient ID 601, the name (Chinese characters) 602, the age 603, and the gender 604 stored in the RAM 203 to the image file stored in the RAM 203 in step S1411, as EXIF information, and stores the lesion image in the RAM 203 (step S1333 in FIG. 13B). When processing for associating patient information ends, the CPU 201 advances the procedure to step S1423.

Steps S1423 to S1426 are similar to steps S8013 to S8016, and thus a description thereof is omitted.

As described above, even when photographing person authentication fails due to a communication failure, the image capture apparatus 101 can easily transmit an authentication request again and obtain patient information after lesion image capturing is completed and associate the patient information and the captured image with each other, by storing an authentication request.

### Other Embodiments

The present invention can be implemented by processing of supplying a program for implementing one or more functions of the above-described embodiments to a system or apparatus via a network or storage medium, and causing one or more processors in the computer of the system or apparatus to read out and execute the program. The present invention can also be implemented by a circuit (for example, an ASIC) for implementing one or more functions.

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2023-115465, filed Jul. 13, 2023 which is hereby incorporated by reference herein in its entirety.

What is claimed is:

1. A medical system that includes an electronic medical record terminal and an image capture apparatus that includes image capture unit, the system comprising:
a specifying unit configured to specify a photographing person of the image capture apparatus;
an obtaining unit configured to obtain patient information regarding a patient whose medical record information is to be displayed on the electronic medical record terminal that is being operated by the photographing person specified by the specifying unit; and
a unit configured to associate a lesion image obtained by the image capture unit performing image capturing and the patient information obtained by the obtaining unit with each other.

2. The medical system according to claim 1, further comprising
a transmitting unit configured to transmit the lesion image obtained by the image capture unit performing image capturing and the patient information obtained by the obtaining unit in association with each other, to an image management apparatus.

3. The medical system according to claim 1, further comprising
a patient information transmitting unit configured to transmit the patient information obtained by the obtaining unit, to the image capture apparatus.

4. The medical system according to claim 3,
wherein the image capture apparatus includes a recording unit configured to record the transmitted patient information and the lesion image obtained by the image capture unit performing image capturing, in association with each other.

5. The medical system according to claim 1,
wherein the specifying unit specifies a photographing person based on information input to the image capture apparatus by the photographing person, and
the obtaining unit specifies a patient whose medical record information is to be displayed on the electronic medical record terminal that is being operated by the photographing person specified by the specifying unit, based on information regarding the photographing person specified by the specifying unit and login information of the electronic medical record terminal, and obtains patient information of the specified patient.

6. The medical system according to claim 5, further comprising
a management unit configured to manage a user for which login authentication was successfully performed in the electronic medical record terminal and a patient whose medical record information is to be displayed on the electronic medical record terminal, in association with each other.

7. The medical system according to claim 6,
wherein the obtaining unit specifies the patient associated with the same user as the photographing person specified by the specifying unit, based on information managed by the management unit, and obtains patient information corresponding to the patient.

8. The medical system according to claim 1,
wherein, in a case where the obtaining unit fails to obtain the patient information before a lesion image is captured, the obtaining unit stores photographing person information specified by the specifying unit, captures a lesion image, and then obtains the patient information based on the photographing person information.

9. The medical system according to claim 1,
wherein the image capture apparatus causes the image capture unit to capture an image of a lesion and generate a lesion image in accordance with an instruction to capture an image of a lesion, and associates the patient information with the lesion image.

10. The medical system according to claim 1, further comprising:
a management apparatus that manages medical record information of patients and information regarding medical workers, and an image management apparatus that manages lesion images of patients.

11. The medical system according to claim 10,
wherein the specifying unit specifies a photographing person based on the information regarding medical workers managed by the management apparatus.

12. The medical system according to claim 11,
wherein the electronic medical record terminal performs login authentication based on the information regarding medical workers that is managed by the management apparatus, and performs control to display the medical record information managed by the management apparatus in accordance with login authentication having been successfully performed.

13. A control method of a medical system that includes an electronic medical record terminal and an image capture apparatus that includes an image capture unit, the method comprising:
specifying a photographing person of the image capture apparatus;
obtaining patient information regarding a patient whose medical record information is to be displayed on the electronic medical record terminal that is being operated by the photographing person specified in the specifying; and
associating a lesion image obtained by the image capture unit performing image capturing and the patient information obtained in the obtaining, to each other.

14. A non-transitory computer-readable storage medium storing thereon a computer program which, when loaded into and executed by a computer provided in a medical system that includes an electronic medical record terminal and an image capture apparatus that includes an image capture unit, causes the computer to perform processes comprising:
specifying a photographing person of the image capture apparatus;
obtaining patient information regarding a patient whose medical record information is to be displayed on the electronic medical record terminal that is being operated by the photographing person specified in the specifying; and
associating a lesion image obtained by the image capture unit performing image capturing and the patient information obtained in the obtaining, to each other.
